(19) United States
(12) Patent Application Publication
(10) Pub. No.: US 2024/0277981 A1
(43) Pub. Date: Aug. 22, 2024

(54) SHUNT WITH OFFSET ANCHOR ARMS

(71) Applicant: EDWARDS LIFESCIENCES CORPORATION, Irvine, CA (US)

(72) Inventors: Rani Abdullah Mahmoudi, Huntington Beach, CA (US); Jean-Pierre Michel Rabbah, Tustin, CA (US); Gabriel Bourche, Irvine, CA (US)

(21) Appl. No.: 18/650,662

(22) Filed: Apr. 30, 2024

Related U.S. Application Data
(63) Continuation of application No. PCT/US2022/048537, filed on Nov. 1, 2022.
(60) Provisional application No. 63/263,755, filed on Nov. 8, 2021.

(51) Int. Cl. A61M 27/00

(52) U.S. Cl. CPC A61M 27/002; A61M 2205/04; A61M 2205/3303; A61M 2205/3327; A61M 2205/3331; A61M 2205/3553; A61M 2205/3561; A61M 2205/502; A61M 2205/8206; A61M 2210/125; A61M 2230/30

(57) ABSTRACT

A shunt device includes a cylindrical barrel portion formed of a plurality of struts arranged in a chevron pattern, a plurality of proximal anchor features emanating from a first axial end of the barrel portion, and a plurality of distal anchor arms emanating from a second axial end of the barrel portion, the plurality of distal anchor arms having a length that is greater than a length of the proximal anchor features.

### RELATED APPLICATION

This application is a continuation of International Patent Application Serial No. PCT/US22/48537, filed Nov. 1, 2022 and entitled SHUNT WITH OFFSET ANCHOR ARMS, which claims priority to U.S. Provisional Patent Application Ser. No. 63/263,755, filed on Nov. 8, 2021 and entitled SHUNT WITH OFFSET ANCHOR ARMS, the complete disclosures of which are hereby incorporated by reference in their entireties.

### BACKGROUND

The present disclosure generally relates to the field of medical implant devices. Certain physiological parameters associated with chambers of the heart, such as fluid pressure, can have an impact on patient health prospects. In particular, high cardiac fluid pressure can lead to heart failure and/or other complications in some patients. Therefore, reduction of pressure in certain chambers of the heart through blood flow shunting can improve patient health in some cases.

### SUMMARY

Described herein are one or more methods and/or devices to facilitate the shunting of blood between chamber(s)/vessel(s) of the heart or other anatomy, and/or the monitoring of certain physiological parameters using certain implant devices. For example, shunt devices are described herein having circumferentially-offset arms and/or chevron strut patters, as well as certain delivery systems and procedures for delivering and deploying the same.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions.

In addition, various features of different disclosed embodiments can be combined to form additional embodiments, which are part of this disclosure. Throughout the drawings, reference numbers may be reused to indicate correspondence between reference elements.

FIG. 1 illustrates human cardiac anatomy in accordance with one or more embodiments.

FIG. 2 illustrates a superior view of a human heart in accordance with one or more embodiments.

FIGS. 3A, 3B, 3C, 3D, 3E, 3F, 3G-1, and 3G-2 show views of a shunt implant in accordance with one or more embodiments.

FIGS. 4A, 4B, and 4C show perspective, exploded perspective, and axial views, respectively, of a shunt implant device having a sensor associated therewith in accordance with one or more embodiments.

FIG. 5 is a block diagram representing a system for monitoring one or more physiological parameters associated with a patient according to one or more embodiments.

FIG. 6 shows a shunt implant device implanted in a coronary sinus tissue wall in accordance with one or more embodiments.

FIG. 7 shows a shunt implant device implanted in an atrial septum in accordance with one or more embodiments.

FIGS. 8A and 8B show a shunt implant device implanted in tissue wall segments having varying thicknesses in accordance with one or more embodiments.

FIGS. 9-1, 9-2, 9-3, 9-4, 9-5, 9-6, and 9-7 provide a flow diagram illustrating a process for implanting a shunt device in accordance with one or more embodiments.

FIGS. 10-1, 10-2, 10-3, 10-4, 10-5, 10-6, and 10-7 provide images of cardiac anatomy and certain devices/systems corresponding to operations of the process of FIGS. 9-1, 9-2, 9-3, 9-4, 9-5, 9-6, and 9-7 in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect. feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Certain standard terms of location are used herein to refer to certain device components/features and to the anatomy of animals, and namely humans, with respect to some embodiments. Although certain spatially relative terms, such as “outer.” “inner,” “upper,” “lower,” “below,” “above,” “vertical,” “horizontal,” “top,” “bottom,” “under,” “over,” “topside,” “underside,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

The present disclosure relates to systems, devices, and methods for shunting blood from a chamber or vessel of, for example, the heart (e.g., the left atrium) to a relatively lower-pressure chamber or vessel (e.g., right atrium, coronary sinus). Such shunting may be considered left-to-right shunting in that it involves the shunting of blood from a left-side chamber/vessel to a right-side chamber/vessel, which can be advantageous for reasons discussed in detail below due to the higher fluid pressures typically experienced on the left (e.g., oxygenated) side of the blood circulation during at least portion(s) of the cardiac cycle. In some implementations, the present disclosure relates to wireframe shunts having anchoring arms that are circumferentially offset with respect to an axis of the barrel of the shunt. Furthermore, embodiments of shunt devices of the present disclosure can include barrel portions that are formed of curved and/or straight chevron-/zigzag-style circumferential/lateral struts, at least some of which may be wishbone-shaped struts, which may accommodate circumferential crimping of the barrel for compressed configuration for delivery. Such shunt implant devices can be configured to hold, and/or may otherwise have associated or integrated therewith, one or more sensor devices for physiological parameter monitoring. The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Certain embodiments of shunt implant devices are disclosed herein in the context of cardiac implant devices and cardiac physiology, which is discussed below in detail to provide context to aid in discussion of aspects of the inventive devices disclosed herein. However, although certain principles disclosed herein are particularly applicable to the anatomy of the heart, it should be understood that shunt implant devices in accordance with the present disclosure may be implanted in, or configured for implantation in, any suitable or desirable anatomy.

### Cardiac Physiology

The anatomy of the heart is described below to assist in the understanding of certain inventive concepts disclosed herein. In humans and other vertebrate animals, the heart generally comprises a muscular organ having four pumping chambers, wherein the flow between chambers and vessels associated therewith is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

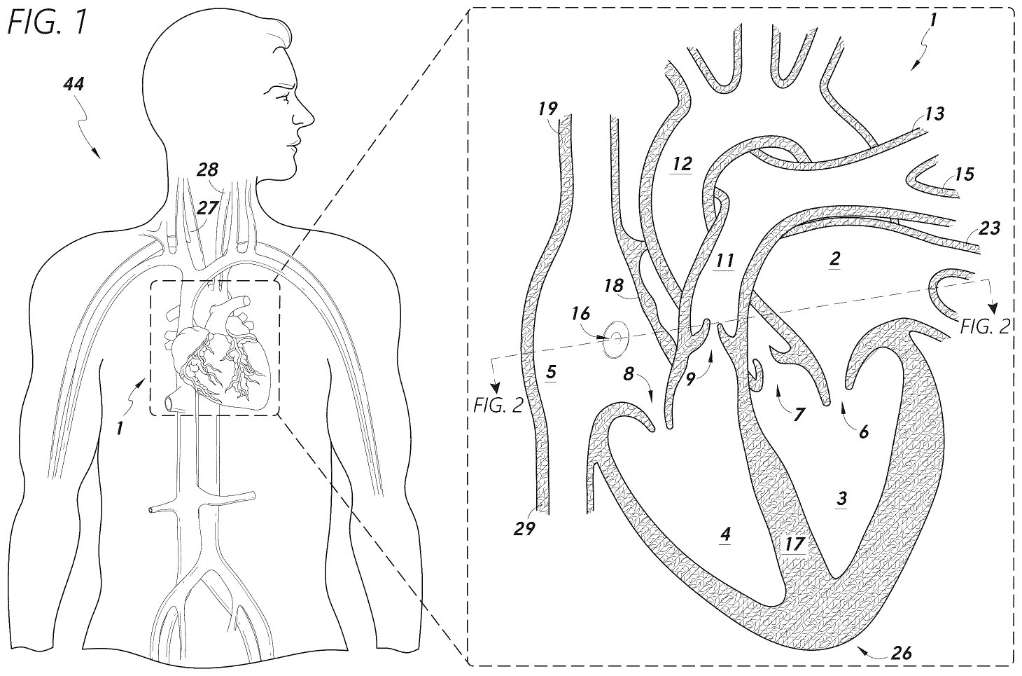

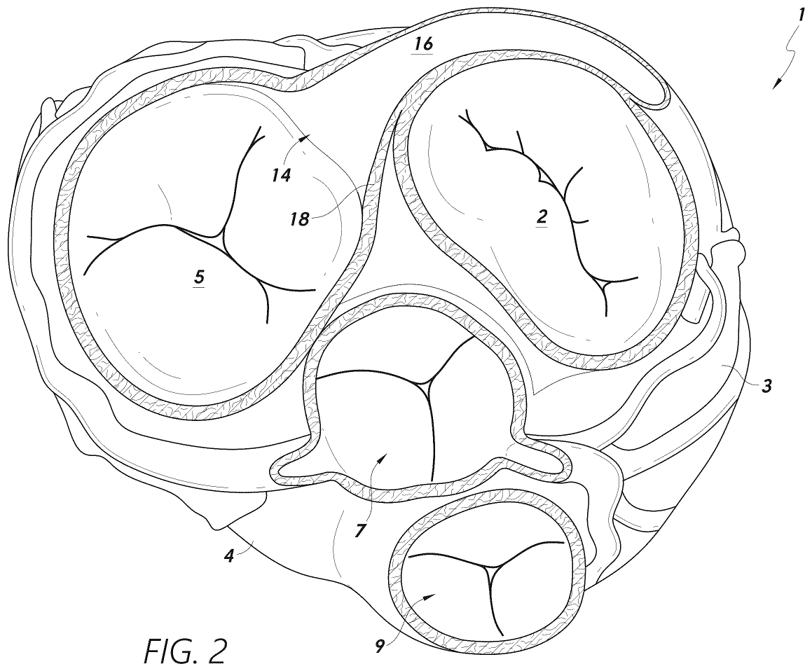

FIGS. 1 and 2 illustrate vertical/frontal and horizontal/superior cross-sectional views, respectively, of an example heart 1 having various features/anatomy relevant to certain aspects of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery 11 via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs.

In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps or leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves may further comprise a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae.

A wall of muscle, referred to as the septum, separates the left-side chambers from the right-side chambers. In particular, an atrial septum wall portion 18 (referred to herein as the “atrial septum,” “atrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 26 of the heart 1 is referred to as the apex and is generally located on or near the midclavicular line, in the fifth intercostal space.

The coronary sinus 16 comprises a collection of veins joined together to form a relatively large vessel that collects blood from the heart muscle (myocardium). The ostium 14 (see FIG. 2) of the coronary sinus 16, which can be guarded at least in part by a Thebesian valve in some patients, is open to the right atrium 5, as shown. The coronary sinus runs along a posterior aspect of the left atrium 2 and delivers less-oxygenated blood to the right atrium 5. The coronary sinus generally runs transversely in the left atrioventricular groove on the posterior side of the heart.

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

The treatment and/or prevention of heart failure (e.g., congestive heart failure) can advantageously involve the monitoring of pressure in one or more chambers or regions of the heart or other anatomy and/or the shunting of some amount of fluid from a problematic high-pressure chamber/vessel to a lower-pressure chamber/vessel. As described above, pressure buildup in one or more chambers or areas of the heart can be associated with congestive heart failure. The monitoring of cardiac pressures can inform shunting procedures, such as with respect to the need or desire for a shunt implant device and/or the particular dimensions and/or configuration of such device.

Without direct or indirect monitoring of cardiac pressure, it can be difficult to infer, determine, or predict the presence or occurrence of congestive heart failure. For example, treatments or approaches not involving direct or indirect pressure monitoring may involve measuring or observing other present physiological conditions of the patient, such as measuring body weight, thoracic impedance, right heart catheterization, or the like. In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain Jung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to be utilized in congestive heart failure diagnostics, prevention, and/or treatment.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. In addition, for patients that have been discharged, such treatments may necessitate remote telemedicine systems.

### Cardiac Pressure Monitoring

The present disclosure provides systems, devices, and methods for guiding the administration of medication relating to the treatment of congestive heart failure and/or other preventative or treatment interventions (e.g., shunt implant device implantation) at least in part by directly monitoring pressure in the left atrium, or other chamber or vessel for which pressure measurements are indicative of left atrial pressure and/or pressure levels in one or more other vessels/chambers, such as for congestive heart failure patients in order to reduce hospital readmissions, morbidity, and/or otherwise improve the health prospects of the patient. For example, embodiments of shunt implant devices disclosed herein can include pressure sensor devices secured to one or more structural features (e.g., sensor holder tabs, arms, etc.) of the shunt implant device for direct pressure monitoring. In some implementations, shunt implant devices of the present disclosure are configured to hold sensor devices along and/or outside of a barrel of the shunt device, such that the sensor device is disposed, trapped, and/or sandwiched between the barrel of the shunt device and a tissue wall in which the shunt barrel/implant is implanted. For example, such devices can be configured to hold the sensor at an orientation that is substantially parallel (e.g., within 15° of parallel) with an axis of the shunt barrel and/or an axis of the opening in the tissue wall (e.g., an axis that is normal to the plane of the tissue wall in the area of the implant device).

Cardiac pressure monitoring in accordance with embodiments of the present disclosure may provide a proactive intervention mechanism for preventing or treating congestive heart failure and/or other physiological conditions. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with embodiments of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe sufficiently. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to embodiments of the present disclosure, normal ventricular filling pressures may advantageously be maintained, thereby preventing or reducing effects of heart failure, such as dyspnea.

As referenced above, with respect to cardiac pressures, pressure elevation in the left atrium may be particularly correlated with heart failure. Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension, which affects approximately 25% to 83% of patients with heart failure, can affect the reliability of pulmonary artery pressure measurement for estimating left-sided filling pressure. Therefore, pulmonary artery pressure measurement alone, as represented by the waveform 124, may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

Left atrial pressure readings may be relatively less likely to be distorted or affected by other conditions, such as respiratory conditions or the like, compared to readings from other chambers/vessels. Generally, left atrial pressure may be significantly predictive of heart failure, such as up two weeks before manifestation of heart failure. For example, increases in left atrial pressure, and both diastolic and systolic heart failure, may occur weeks prior to hospitalization, and therefore knowledge of such increases may be used to predict the onset of congestive heart failure, such as acute debilitating symptoms of congestive heart failure.

Embodiments of the present disclosure that include integrated pressure sensors with shunt implant devices can provide for direct left atrial pressure monitoring, which can provide a mechanism to guide administration of medication to treat and/or prevent congestive heart failure. Such treatments may advantageously reduce hospital readmissions and morbidity, as well as provide other benefits. An implanted pressure sensor in accordance with embodiments of the present disclosure may be used to predict heart failure up two weeks or more before the manifestation of symptoms or markers of heart failure (e.g., dyspnea). When heart failure predictors are recognized using cardiac pressure sensor embodiments in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

### Shunt Implant Devices with Offset Arms and/or Chevron Struts

As referenced above, the treatment of certain cardiac conditions can involve the implantation of devices designed to shunt blood from one chamber or vessel of the heart to another. Disclosed herein are novel wireframe shunt implant devices including chevron-style struts configured to allow for the devices to be crimped circumferentially around a barrel thereof and transported to a target treatment site in a configuration in which the barrel of the shunt device is crimped to a relatively compressed configuration/diameter within a delivery sheath or catheter and expanded upon deployment from the delivery sheath/catheter. Such implant devices can be configured with vertically and/or circumferentially/angularly offset arms on opposite axial ends of the shunt/barrel, which may aid in the ability to crimp the device to a relatively small profile, as well as improve secure positioning within the target tissue wall.

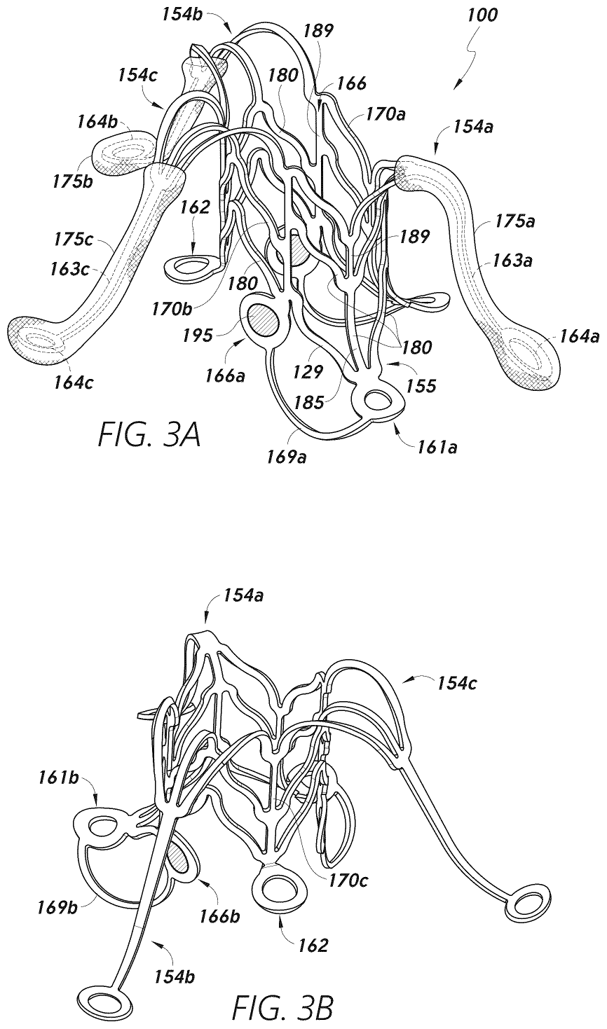

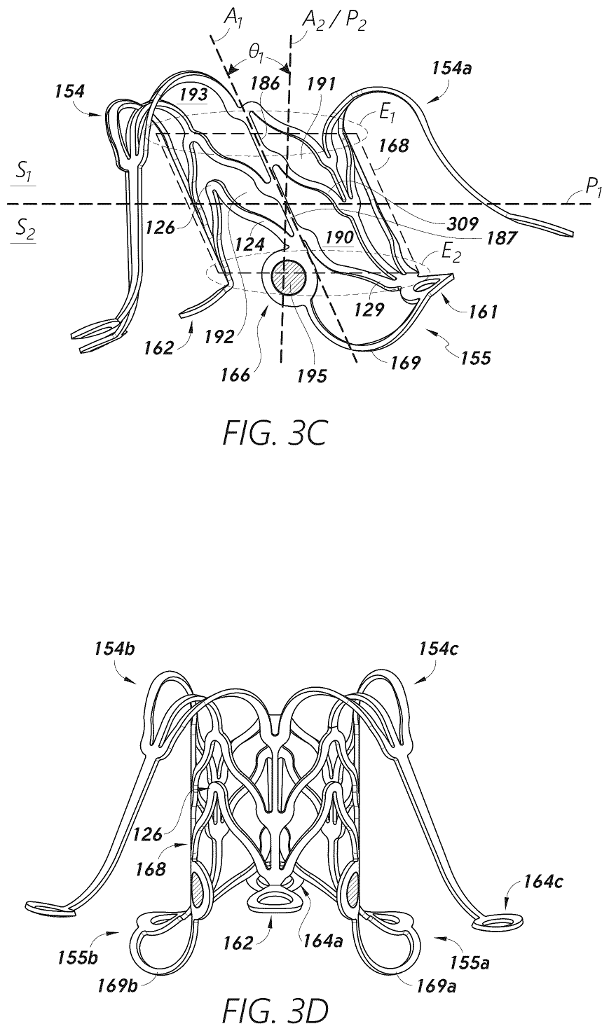

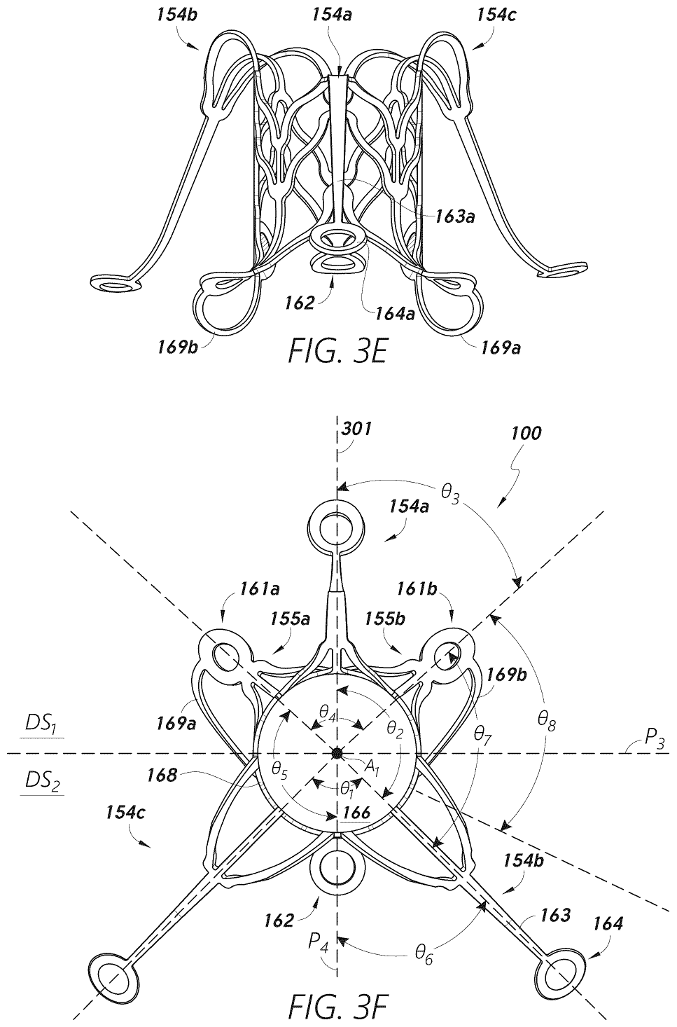

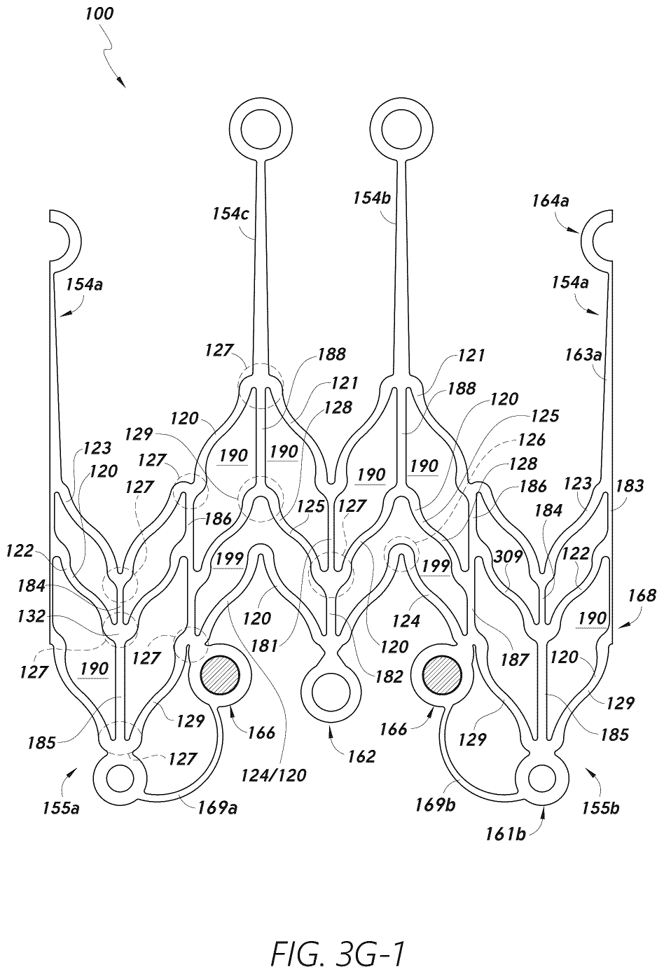

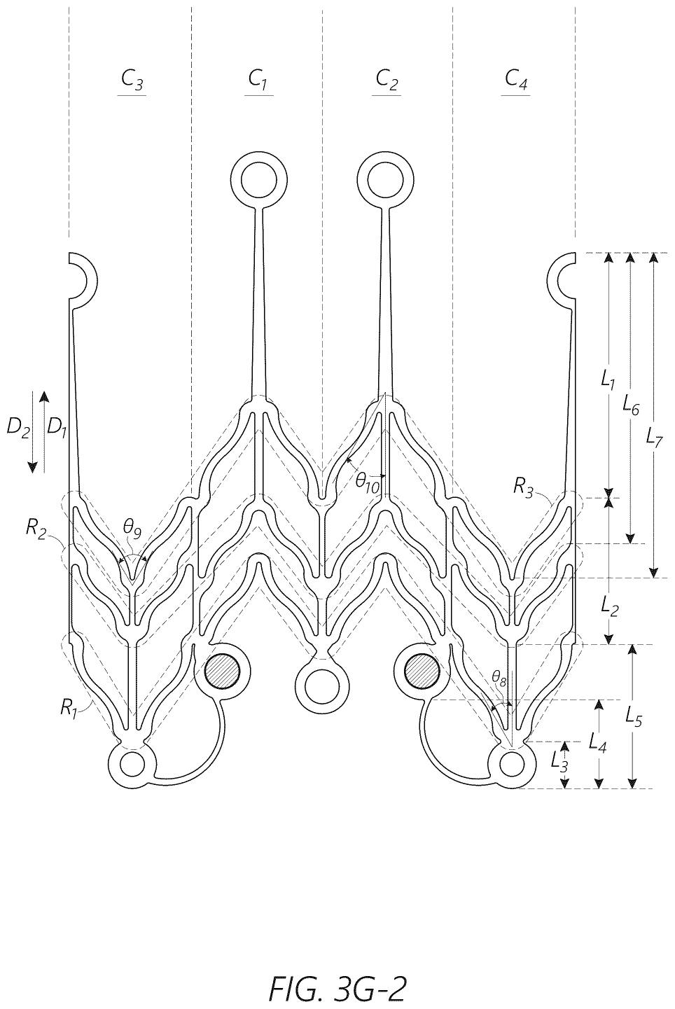

FIGS. 3A-3G show views of a shunt implant device 100 in an expanded configuration (with the exception of FIGS. 3G-1 and 3G-2, which show a flat, rolled-out view/configuration) in accordance with one or more embodiments of the present disclosure. In particular, FIG. 3A and 3B show perspective views of the device 100, while FIG. 3C-3E show side views. FIG. 3F shows a top-down axial view looking through the central flow tube/lumen 166 formed by the barrel 168 of the shunt device 100 along an axis A, of the barrel 168. When expanded, the barrel 168 and central flow lumen 166 of the shunt 100 can define a generally circular or oval opening and fluid passageway, as seen from above in FIG. 3F, wherein the barrel 168 is configured to hold the sides of a puncture/hole in a tissue wall open and form a blood flow path between chambers/vessels on opposite sides of the tissue wall (e.g., coronary sinus and left atrium). The central flow lumen 166 is partly formed by side walls 170 of the barrel structure 168 of the shunt 100.

The shunt structure 100 can include a plurality of distal anchor arms 154 emanating from a distal axial end E₁ of the barrel 168. The distal arms 154 may be relatively long and have tissue contact pads/feet 164 associated with distal ends thereof, wherein such pads/feet 164 are configured to contact a surface of a tissue wall in which the shunt 100 is implanted. The illustrated embodiment includes circular/eyelet-type contact pads/feet 164, although it should be understood that other-shaped tissue-contact pads/feet may be implemented in connection with embodiments of the present disclosure. The pads/feet 164 may provide a relatively wide/spread-out area relative to the relatively narrow elongated strut/arm portion 163, which may advantageously distribute contact force/pressure exerted by the shunt 100 on the biological tissue wall/surface over a relatively wider area.

The shunt structure 100 can further include a plurality of proximal anchor features/arms 155 emanating from a proximal axial end E₂ of the barrel 168. In some embodiments, the proximal arms 155 include a primary tissue contact/pad 161 and a secondary tissue contact/pad 166, which may be angularly offset from the primary contact pad 161 by an amount θ₈ with respect to the axis At of the barrel 168, and vertically/axially offset therefrom by some amount as well, as shown. For example, the angular offset θ₈ of the primary 161 and secondary 166 contact pads of the proximal anchor features may be between 45-90°, such as between 60-80°, such as about 70° in some embodiments. The proximal anchor features 155 can be configured to deflect and/or be deflected outwardly from the barrel 168, as shown. The proximal anchor features 155, in whole or in part, may be considered proximal anchoring means, wherein such means may have any of the anchoring configurations illustrated and/or disclosed herein, including a combination of struts, arms, contact pads, and features/struts connecting the same.

The primary contact pad 161 may be coupled mechanically to the secondary contact pad 166 by a vertical offset arm 169, which may provide added mechanical stability for the anchor arm 155 and/or increase the tissue contact surface area of the proximal anchor arm 155. For example, the vertical offset arm 169 can be configured to contact the tissue wall and serve to increase the footprint/area of contact covered by the anchor feature/arm 155 to further secure the shunt structure 100 to the tissue wall. The primary contact pad 161 may be vertically offset from the secondary contact pad 166 by an amount/distance Ly, as shown in FIG. 3G. In some implementations, the proximal anchoring features/means 155 may be considered to consist of the primary contact pad 161, the secondary contact pad 166, the connecting arm/strut 169, the longitudinal strut 185 coupled to the primary contact pad 161, one or both of the lateral/circumferential struts 129 connected to the primary contact pad 161 from the side, or any combination thereof. As illustrated, the connecting/offset arms/struts 169 can be curved or bowed away from the axial center of the barrel 168 and/or from the contact pad 161, contact pad 166, and/or strut 129. Such bowing/curving can increase the tissue contact area/stability of the shunt 100.

In some embodiments, one or more of the tissue contact pads 161, 164, 166 may have associated therewith certain visual marker features configured to provide increased visualization under imaging, such as ultrasound, x-ray, or other imaging modality. For example, the tissue contact pads and/or other features of the shunt structure 100 may include certain visual marker bands, studs, or the like comprising echogenic or other imaging-enhancement characteristics. In the illustrated embodiment, the secondary contact pads 166 include marker studs 195 that may comprise an echogenic material, such as metal (e.g., tantalum) or other material configured to be relatively identifiable/visible under an imaging mechanism. The illustrated circular form of the various tissue contact pads associated with the respective anchor arms can facilitate a process for implementing the visual marker bands/studs therein, such as through a coining process or similar process. For example, a marker stud 195 may be press-fit and/or melted/formed within the central opening/aperture of a contact pad/foot, such as the secondary contact pads/feet 166. Enhanced visualization features of the shunt implant device 100 can aid in intraoperative placement of the implant 100 in the target anatomy.

The shunt structure 100 and/or barrel portion/structure 168 thereof can be defined at least in part by an arrangement of relatively thin struts 180 that form an array of cells/openings 190 (e.g., parallelogram-shaped or other-shaped). For example, some or all of the shunt structure 100 can be formed by super-elastic struts that are capable of compression into a delivery catheter and subsequent expansion back to the relaxed shape as shown in FIGS. 3A-3F. Formation of the shunt 100 using a plurality of interconnected struts forming cells 190, 199 therebetween can advantageously provide desirable flexibility of the shunt, which enables compression and subsequent expansion at the implant site. The interconnected struts 180 around the barrel 168 forming the central flow lumen 166 can provide a cage-type structure that provides sufficient stability/integrity to hold the tissue at the puncture site open. The struts 180 can be arranged in an interconnected pattern that omits any sharp corners or points, particularly along a first/distal end E₁ or second/proximal end E₂ of the barrel 168, which might snag tissue when the shunt is being manipulated/advanced through/within the puncture.

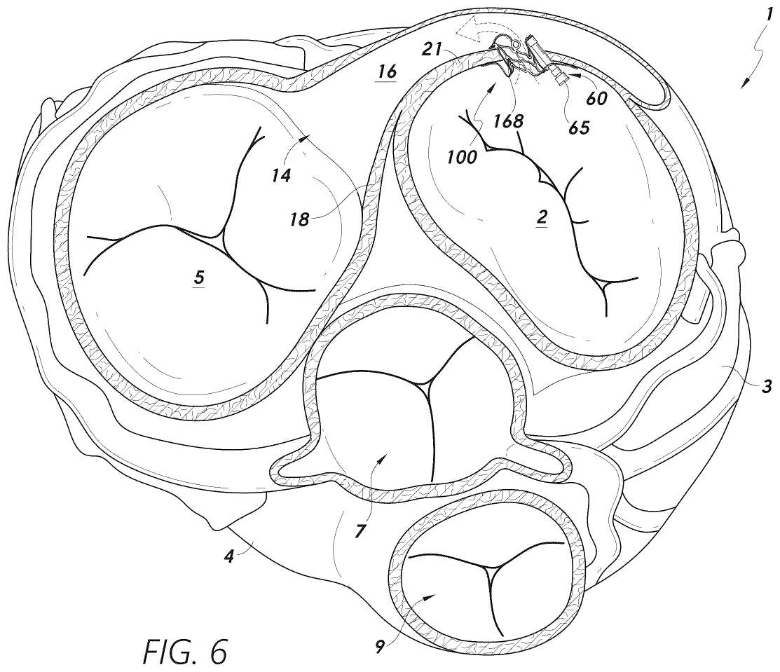

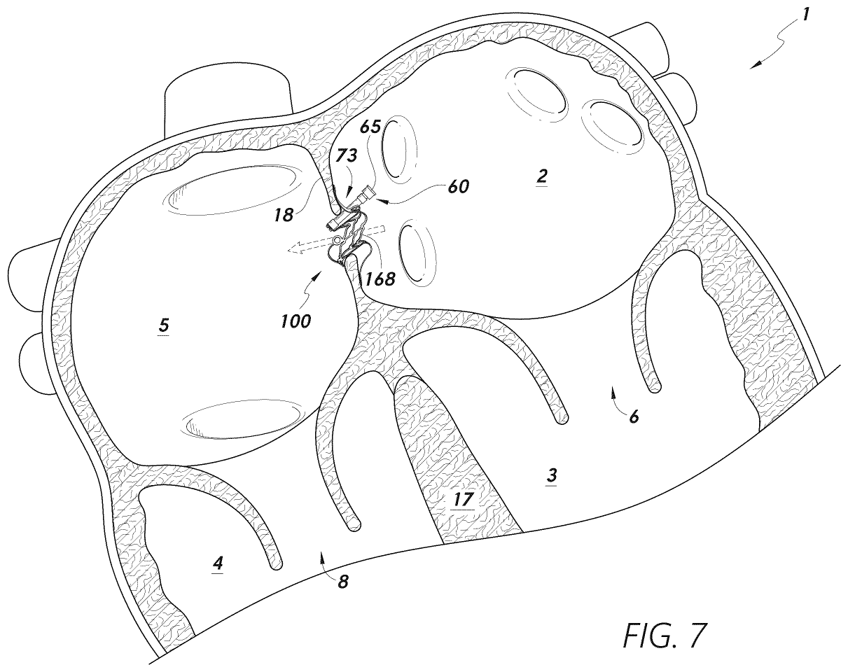

The side walls 170 of the barrel structure 168 together define a tubular lattice that forms a channel/barrel 166, 168 that is angled with respect to a tissue plane P₁ associated with the shunt structure 100. The plane P₁ may be orthogonal to the axis A₁ of the barrel portion 168 of the shunt structure 100 and/or may be substantially parallel with (e.g., on/within) a tissue wall in which the shunt structure 100 is implanted. That is, when the shunt structure 100 is implanted in a tissue wall (not shown in FIGS. 3A-3G; see FIGS. 6-7), the axis A₁ of the barrel 168 may be askew/angled with respect to a line/plane A₂ that is normal to the tissue wall surface; it should be understood that description herein of shunt axes may be understood to refer to an axis/line that is substantially normal to a tissue-engagement plane (e.g., plane P₁ shown in FIG. 3C), even in embodiments/cases in which the shunt barrel has a true axis A₁ that is angled/canted with respect to the tissue-engagement plane P₁, as in FIG. 3C. Description herein of axial sides of an implant structure can be understood to refer to different sides of the tissue-engagement plane P₁. The plane P₁ may be aligned (e.g., within 15° of exact alignment) with at least some of the lateral/circumferential struts of the barrel/conduit portion 168 of the shunt structure 100.

The axial/longitudinal/vertical struts 181-188 of the barrel 168 may generally align/deflect at a slight angle θ, from a perpendicular axis/plane A₂/P₂ through the central flow channel 166. That is, as seen in FIG. 3C, an imaginary reference axis/plane A₂/P₂ may be drawn generally perpendicular to the horizontal reference plane P₁, such that an angled axis A₁ is defined by the axis of the barrel 168. The angle θ₁ may be between 30-60°, such as about 45°, or between 15-45°, such as about 30°. The horizontal reference plane P₁ is generally defined by the tissue wall (e.g., wall separating the coronary sinus and left atrium) in which the shunt 100 is placed; though, of course, the wall may generally not be simply planar and the tissue plane P₁ may simply represent a planar approximation of the wall in the area of the implantation. Although oriented at an angle θ₁, the opening formed by the central flow barrel 168 may be generally perpendicular to the tissue plane P₁ due to the widening of the barrel 168 at the ends E₁, E₂ thereof, such as may be due in part to the outward extension and curvature of the distal 154 and proximal 155 arms. That is, the angled barrel 168 can be wide and short enough such that shunting occurs as if the flow channel 166 was perpendicular to the tissue plane P₁.

FIG. 3F is a view looking down an angled axis A₁ of the central flow lumen 166 of the expanded shunt 100. The axis A₁ defines the “tilt” of the expanded shunt 100, in that it defines the angle that the barrel 168 makes with the horizontal reference plane P₁, which again lies generally in the plane of the tissue wall through which the shunt passes. It can thus be seen that the struts 180 of the barrel 168 define a tubular or cylindrical lattice, even if the struts that form the barrel do not form a contiguous wall surface (e.g., there are open cells 190, 199). The tilt of the expandable shunt 100 facilitates collapse into the delivery catheter, and then expansion of the flanges/arms 154, 155 on both sides of the target tissue wall. In some implementations, the central barrel 168 remains essentially unchanged between the collapsed and expanded states of the shunt 100. However, the curved/undulated chevron design of the circumferential/lateral struts 120 can allow for the barrel 168 to be radially compressed for transport, thereby allowing for a further reduced compressed profile with respect to the diameter of the barrel 168. Although shown in FIG. 3F as having a circular cross-sectional shape, it should be understood that the barrel 168 can have an oval-shaped cross-section, or other shape. The circumferential/lateral struts 120, as shown, can be arranged in a chevron and/or zigzag pattern.

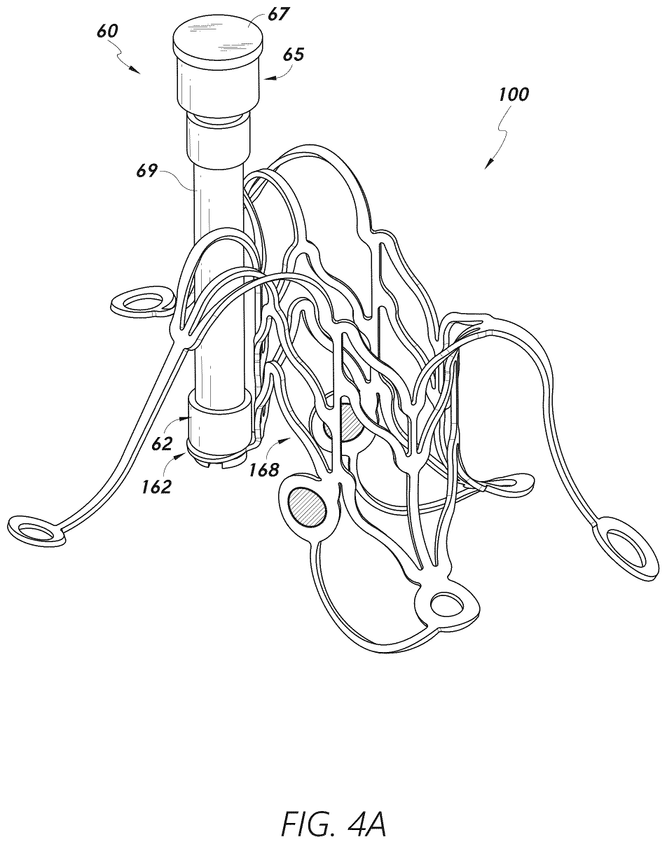

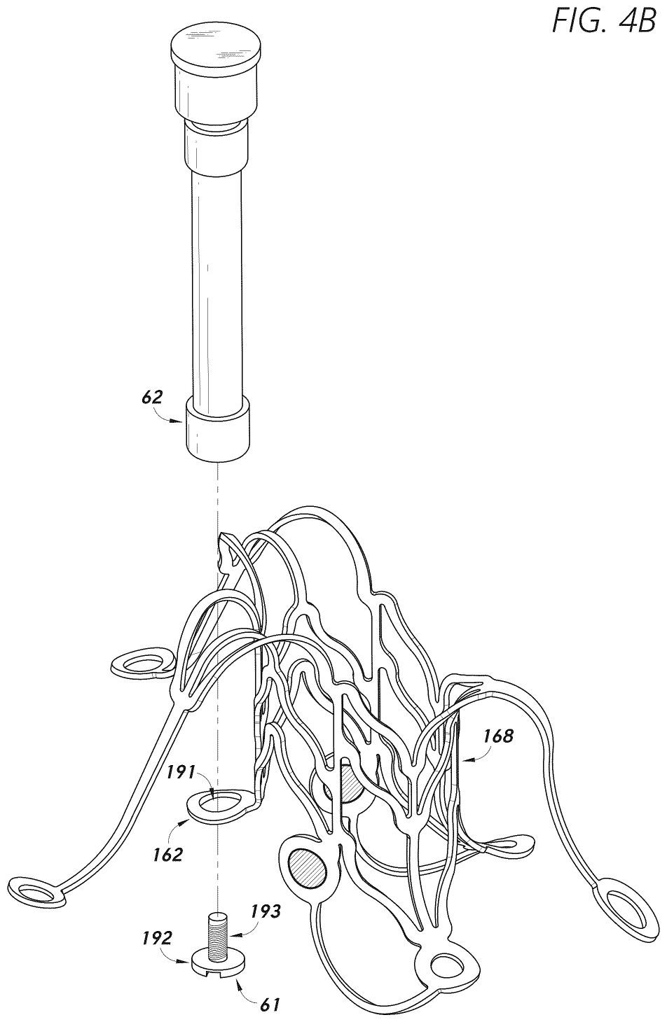

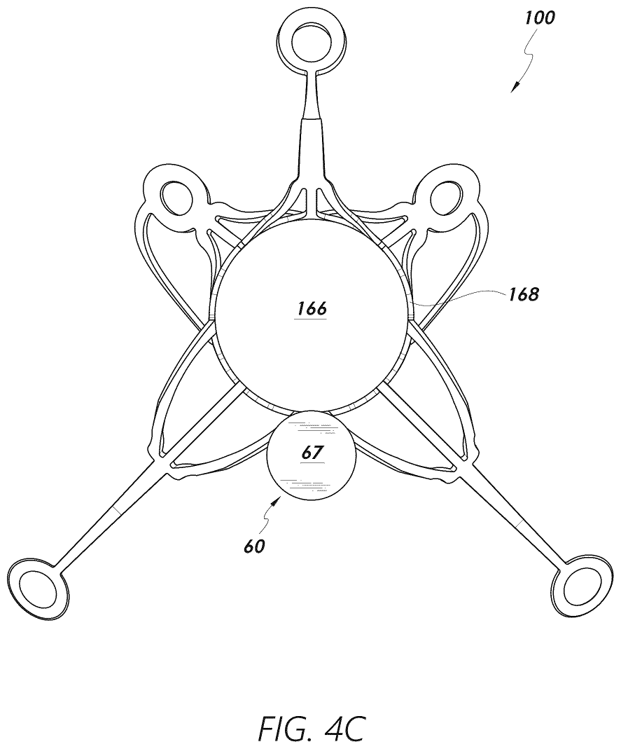

The barrel 168, as referenced above, is configured to be radially crimped to a collapsed or crimped state (see, e.g., image 1006A of FIG. 10-4) for introduction into the body on/in a delivery catheter and radially expandable to an expanded state for implanting the shunt 100 at a desired location in the body (e.g., atrial septum or wall separating coronary sinus and left atrium). The shunt 100 can be made of a plastically-expandable material that permits crimping of the struts thereof to a smaller profile for delivery and expansion, wherein such expansion may be performed using an expansion device (e.g., balloon of a balloon catheter) and/or through self-expansion caused by shape memory characteristics. For embodiments utilizing self-expanding struts/structure, the shunt 100 can be crimped to a smaller profile and held in the crimped state with a restraining device, such as a sheath, covering the compressed shunt. When the shunt 100 is positioned at or near the target site, the restraining device can be removed to allow the shunt 100 to self-expand to its expanded, functional size shown in FIGS. 3A-3G.

The shunt 100 comprises a plurality of axial/vertical struts 181188 connected by a number of rows R₁, R₂, R₃ of circumferential/lateral curved struts 120, some of which have a wishbone shape/form, to form a generally tubular structure. A lower/outflow end E₂ of the shunt 100 includes a circumferential row R₂ of wishbone-, or crown-shaped struts 124, 129, wherein such struts are arranged as circumferential columns C₁₋₄ separated by vertical/axial struts 181, 183, and 186. Generally, as shown in FIGS. 3G-1 and 3G-2 (FIGS. 3G-1 and 3G-2 are collectively referred to herein as FIG. 3G), the wishbone struts 129 may be open in a first axial/longitudinal direction D₁, whereas the wishbone struts 124 are open in an opposite direction D₂. Furthermore, in some embodiments, the wishbone struts 129 may be supported/attached at an apex 127 thereof, on an open side of the apex 127, by/to an axial/vertical strut 185, which may provide desirable stability for the barrel 168 in such areas. Conversely, in some embodiments, the wishbone struts 124 may be unsupported/unattached at an apex 126 thereof by/to an axial/vertical strut, which may provide desirable flexibility for the barrel 168 in such areas.

An upper/inflow end E₁ of the shunt 100 includes a circumferential row R₃ of wishbone-, or crown-shaped struts 121, 123, wherein such struts are arranged in the circumferential columns C₁₋₄ separated by the vertical/axial struts 181, 183, and 186. Generally, as shown in FIGS. 3G-1 and 3G-2, the wishbone struts 123 may be open in the first direction D₁, whereas the wishbone struts 121 are open in the opposite direction D₂. Furthermore, in some embodiments, the wishbone struts 123 may be supported/attached at an apex 127 thereof, on a side opposite an open side of the apex 127, by/to an axial/vertical strut 184, which may provide desirable stability for the barrel 168 in such areas. In addition, in some embodiments, the wishbone struts 121 may be supported/attached at an apex 127 thereof, on an open side of the apex 127, by/to an axial/vertical strut 188, which may provide desirable flexibility for the barrel 168 in such areas.

The shunt 100 can include an intermediate circumferential row R₂ of wishbone-, or crown-shaped struts 122, 125, wherein such struts are arranged in the circumferential columns C₁₋₄ separated by the vertical/axial struts 181, 183, and 186. Generally, as shown in FIGS. 3G-1 and 3G-2, the wishbone struts 122 may be open in the first direction DI, whereas the wishbone struts 125 are open in the opposite direction D₂. Furthermore, in some embodiments, the wishbone struts 122 may be supported/attached at an apex 127 thereof, on both sides of the apex 127, by/to axial/vertical struts 184, 185, which may provide desirable stability for the barrel 168 in such areas. In addition, in some embodiments, the wishbone struts 125 may be supported/attached at an apex 129 thereof, on a side opposite an open side of the apex 127, by/to an axial/vertical strut 188, which may provide desirable flexibility for the barrel 168 in such areas.

The cells/spaces defined between the wishbone struts 124 and 125 of adjacent rows R₁, R₂, respectively, can be chevron-shaped, pointed upward (in direction D₁). The identified cells 199 are closed chevron cells. For example, generally, the columns C₁₋₂ of struts can form closed cells, including full and broken chevron cells pointing in the first direction D₁, whereas the columns C₃₋₄ of struts can form broken chevron cells pointing in the second direction D₂, as shown. “Broken” chevron cells, as described and shown, can refer to chevron cells that are bisected at an apex area thereof by a vertical/axial strut (e.g., struts 188), such that two diamond (e.g., curved/wavy diamond, as shown) cells are formed.

The shunt 100 further comprises a plurality of distal anchor means 154 emanating from the first axial end E, of the barrel 168, wherein the anchor arms/means 154 are configured to deflect outwardly from the barrel 168, as shown. The distal anchor means 154 may comprise anchor arms, as shown. In some embodiments, the distal anchor arms/means 154 comprise elongated struts/arms 163 terminating at distal ends thereof in respective tissue contact pads 164 configured to contact and/or press against the tissue wall in which the shunt 100 is implanted. The distal anchor arms 154 may advantageously be longer than the proximal anchor arms 155, which may provide desirable flexibility with respect to fitting different sizes and/or configurations of anatomies. In some implementations, the distal anchoring arms/means 154 may be considered to consist of the tissue contact pad 164, the elongated strut 163, one or both of the lateral/circumferential struts 121 (which may be considered a single wishbone strut) connected to the elongated strut 163 from the side, the longitudinal strut 188 coupled to the elongated strut 163, or any combination thereof.

The sets of distal long arms 154 and proximal short arms 155 can have shape memory configured to curl outward from the barrel 168 and/or axis A₁ when deployed and/or expanded. Portions of such arms/anchor means, in the expanded configuration shown, can project approximately radially away from the imaginary reference axes A₂ and/or A₁ and/or towards an axial center of the barrel 168 (e.g., the axial position of the plane P₁). The three long flanges/arms 154 may project away from each other, as do the two proximal pads/flanges/arms 155. The barrel 168 generally has a tilted/canted orientation relative to the tissue plane P₁ and/or line/plane A₂/P₂ normal/orthogonal to the tissue plane P1, as indicated by the angle θ₁.

The various anchor means/arms of the shunt device 100 may be angularly distributed about the axis A₁ of the shunt 100 and/or barrel 168 in any suitable or desirable manner. For example, as best illustrated in the axial view of FIG. 3F, the distal/long anchor arms 154 may be associated with a first end E1 of the barrel 168 and may be angularly distributed in some manner about the perimeter/circumference of the barrel 168, wherein the arms 154, in the expanded configuration, project away from the barrel 168 and/or axis A₁.

In the illustrated embodiment of FIGS. 3A-3G, the first/distal end E₁ has three long anchor arms 154 associated therewith, namely anchor arms 154a, 154b, and 154c. Although embodiments of the present disclosure are illustrated and described in some contexts as having three distal and/or long anchor arms 154, it should be understood that embodiments of the present disclosure may have any suitable or desirable number of long and/or distal anchor arms. Such arms 154 may be evenly, or about evenly (e.g., within 45° of even distribution), distributed angularly around the barrel 168. In some embodiments, the distal/long anchor arms 154 comprise a first long anchor arm 154a that is angularly positioned between the proximal/short anchor arms 155 on a same diametrical side DS₁ as the tissue contact pads 161 of the short anchor arms 155. Although the proximal arms 155 are described and illustrated as relatively short compared to the distal arms 154 in some contexts herein, it should be understood that in some embodiments, the proximal arm(s) 155 associated with the side E₂ opposite the side E associated with the distal arms 154 can be longer than illustrated and/or equally long relative to the distal arms 154. The diametrical sides DS₁, DS₂ are defined and referenced herein to aid in the description of the angular/circumferential positions of the various features and/or arms of the shunt 100. For example, as used herein, the diametrical sides DS₁, DS₂ may be opposite sides of an axial plane P₃ in which the axis A₁ lies and is perpendicular/orthogonal to a radial ray 301 that is aligned with the first distal arm 154a, as illustrated in FIG. 3F. The ray 301 may be aligned with a line of symmetry of the shunt. That is, with respect to the axial view of FIG. 3F, the shunt 100 may be symmetrical, wherein the line of symmetry 301 bisects one of the distal anchor arms 154, but does not bisect a plurality of other distal anchor arms 154 or the proximal anchor features/arms 155.

In addition to the first arm 154a, which is disposed on the first diametrical side DS₁ of the barrel 168, the shunt 100 may include two additional distal/long arms 154b, 154c positioned on a second diametrical side DS₂. As referenced above, the distal arms 154 may be evenly angularly distributed about the barrel 168 in some embodiments. That is, in some embodiments, the angles θ_{1, θ2} between the distal arms 154 may be substantially equal, with about 120° of separation between them. However, in certain other embodiments, as illustrated in FIGS. 3A-3G, the first arm 154a may be angularly separated from the second and third arms 154b, 154c by a greater amount than the second and third arms 154b, 154c are separate from each other. For example, as shown in FIG. 3F. the first arm 154a may be angularly separated from both of the second and third arms 154b, 154c by an angle θ₂ that is greater than 120°, such as an angle between 120-150°. For example, as shown in FIG. 3F, the first arm 154a may be separated from the second and third arms 154b, 154c by an angle θ₂ that is about 135°. In such embodiments, the second and third arms 154b, 154c, which are positioned on a diametrical side DS₂ that is opposite the first arm 154a and the proximal arms 155 (e.g., tissue contact pads 161), may be separated angularly from one another by an angle θ, that is less than 120°. For example, as shown in FIG. 3F, the second and third distal arms 154b, 154c may be separated by an angle θ₁ that is between 75-105°, such as about 90°.

In some embodiments, the shunt includes a sensor-holder feature 162, which may have a circular, washer-type shape/form, or may have any other structure of form configured to be leveraged to secure a sensor device (e.g., pressure sensor) thereto and/or to the shunt 100. The sensor-holder 162 can be configured to deflect, or be deflected, outwardly from the barrel 168, as shown. In some embodiments, the proximal sensor holder 162 may be angularly/circumferentially positioned between the second and third distal long anchor arms 154b, 154c, as shown. For example, the sensor holder 162 may be angularly positioned opposite of the first distal arm 154a (e.g., 180° separation). The first distal arm 154a may have an equal angular separation θ₃from the first 155a and second 155b proximal short arms (and/or contact pads 161a, 161b of the proximal anchor features/means 155) in some embodiments. For example, the angular distance 63 between the first distal arm 154a and each of the proximal arms 155 may be between 30-60°, such as about 45°, 50°, or any angle therebetween. Therefore, the angular separation between the two proximal arms 155a, 155b (and/or contact pads 161a, 161b) may be between 60-120°, such as about 90°, 100°, or any angle therebetween.

As can be seen in FIG. 3F, each of the distal long arms 154 may be angularly offset from each of the proximal short arms 155. Such angular offset between proximal and distal arms may serve to secure the shunt 100 to the tissue wall in which is implanted in a manner as to resist post-implantation tilting, rotating, or other dislodging, migration, and/or undesirable movement of the shunt 100 from its desired implanted position and/or orientation.

For example, the pressure contact points associated with the feet/pads 164 of the distal arms 154 on the distal side of the tissue wall in which the implant is disposed may be angularly offset from the tissue contact pressure points/areas associated with the feet/pads 161 of the proximal arms 155, thereby distributing contact pressure over a wider area of the tissue wall in which the implant is disposed. The angular offsetting of the proximal arms 155, distal arms 154, and/or sensor holder 162 can further provide structural balance to the shunt structure 100, thereby improving stability and secure attachment of the shunt device 100.

The angular offset θ₅ between the proximal arms 155 and the sensor holder 162 may be greater than the distance θ₄ between the proximal anchor arms 155. For example, the angle of separation θ₅ may be between 250-280°, such as about 270°, 280°, or any angle therebetween. In some embodiments, with respect to the axial view of FIG. 3F, the shunt 100 may be symmetrical about a plane P₄ that is aligned with the radially-projecting ray 301 aligned with the first distal anchor arm 154a. In some embodiments, such plane P₄ may further be aligned with and/or cut through the sensor holder 162. The second 154b and third 154c distal anchor arms may both/each be angularly offset from an adjacent one of the proximal anchor arms 155 by an angle θ7 that may be between 80-100°, such as about 90°, or between 85-90°. That is, moving angularly around the circumference/perimeter of the barrel 168, the distal anchor arms 154b, 154c and the proximal anchor arms 155a, 155b (e.g., contact pads 161a, 161b) may be substantially evenly distributed, such that an approximately 90°/right angle separates the arms, wherein the two proximal anchor arms 155a, 155b are positioned adjacent to one another on a first diametrical side DS₁ of the barrel 168, and the distal anchor arms 154b, 154c are positioned adjacent to one another on an opposite diametrical side DS₂, as shown.

FIG. 3G shows a flattened, rolled-out view of the expandable shunt 100 as if the shunt 100 had been severed down the midline of the first distal/long anchor arm 154a and the aligned portion of the barrel 168. This view illustrates orientations of the curved diamond-and chevron-shaped cells 190 and struts 120 in the side walls 170. With respect to the illustrated orientation of FIGS. 3G-1 and 3G-2, the struts 120 and cells 190 of the shunt structure 100 may have a zigzag configuration moving laterally/circumferentially around the barrel 168 of the shunt structure. For example, with respect to the particular illustrated orientation of FIG. 3G in which the direction towards the top of the page is referred to as an ‘upward’ direction and the direction towards the bottom of the page is referred to as a ‘downward’ direction, moving from the first distal/long arm 154a laterally towards the proximal/short anchor arms 155a, 155b, the struts 120 and cells 190 may be angled/deflected downward. Moving laterally from the proximal anchor arms 155b, 155a towards the second 154c and third 154b distal anchor arms, respectively, the struts 120 and cells 190 may generally be angled in the upward direction. Moving laterally from the second 154b and third 154c distal/long anchor arms towards one another and/or towards the sensor holder 162, the struts 120 and cells 190 may again be deflected/angled downward. Such zigzag pattern of the struts and cells can produce the desired chevron pattern disclosed herein, which allows for barrel compression and produces the canted barrel shape in the expanded configuration. This beneficial juxtaposition of the oppositely-tilted struts/cells can allow for the super elastic material making up the struts to easily flex into an elongated delivery shape/configuration.

The thickness of the struts 120 can be at least 0.2 mm, preferably between 0.2-0.3 mm. In some embodiments, the apertures/eyelets associated with the terminal ends of the anchor arm(s) 154, 155 can define a buckle form that is configured to be engaged by actuating rod(s) used to deploy the respective anchor arm(s). Although illustrated as circular/oval in shape, it should be understood that the terminal feet/pads of one or more of the anchor arms may be rectangular with respect to the periphery thereof and/or aperture associated therewith. It should be understood that the various struts that form the shunt 100 can advantageously be fabricated by laser-cutting a nitinol tube. The tube may have a wall thickness of between about 0.1-0.3 mm, and preferably about 0.2 mm.

The shunt 100, as described in detail above, includes distal long arms 154 and proximal short arms 155. The use of long arms on one or more sides of the shunt 100 can advantageously provide a shunt structure that provides flexibility with respect to anchoring of such anchor arms. For example, the longer the anchor arms 154, the greater the range of tissue wall thicknesses, topologies, shapes, and/or other characteristics the anchor arms may be able to accommodate. That is, the additional length of the arms 154 can allow for a range of bending of the arm to accommodate placement of the contact foot/pad 164 associated therewith at a suitable or desirable position with respect to the tissue wall. Alternatively, the short proximal arms 155 may be less flexible and able to accommodate varying tissue wall thicknesses, topologies and/or other characteristics. However, when the implant device 100 is placed in a wall separating the coronary sinus from the left atrium, the side E₂ of the shunt structure configured to be disposed in the coronary sinus may have relatively less spatial area available for accommodating anchor arms. Therefore, it may be desirable for the anchor arms associated with one side E₂ of the shunt 100 to be relatively short to be suitable for the reduced volume/space associated with the coronary sinus or other chamber/vessel in which it may be placed, whereas the opposite side of the shunt 100 may be disposed at least partially within a relatively larger chamber/vessel, such as the left atrium, and therefore the anchor arms 154 associated with such side E₁ may suitably be relatively longer to allow for the desired anchoring flexibility provided by such anchor arm length.

As can be seen in FIGS. 3G-1 and 3G-2, which shows a flattened-/rolled-out view of the shunt 100, the lateral/circumferential struts 120 may have a curved/corrugated shape, such as in S-curve shape. However, it should be understood that embodiments the present disclosure may include circumferential/lateral struts that are substantially straight with respect to at least portions thereof. Each of the lateral/circumferential struts 120 may be joined at one or more ends thereof to a longitudinal strut that runs substantially parallel with the axis A₁ of the barrel 168 and/or runs predominantly longitudinally with respect to the orientation of the barrel 168. For example, each of the lateral/circumferential struts 120 may be joined with a longitudinal strut at a joint 127 (which may be considered an apex portion/joint of certain wishbone struts of the shunt 100 with respect to some joints of the barrel), wherein the struts 120 may be configured to bend in a manner such that the angle between the strut and the respective longitudinal strut to which it is connected/joined is decreased to achieve a compressed configuration in which the barrel 168 has a reduced diameter, circumference, and/or other dimension allowing for a compressed delivery configuration for placement in the delivery catheter or similar system/device. That is, in the delivery configuration, the respective angles θ₁₀ between the circumferential/lateral struts 120 and the axis of the barrel 168 and/or longitudinal strut to which the lateral/circumferential strut is joined/connected may be relatively small, wherein when expanding to the expanded configuration shown in FIGS. 3A-3F, the angle θ₁₀ of the respective joints 127 between the lateral/circumferential struts 120 and the corresponding longitudinal struts (e.g., struts 181-188) may be increased, such that the barrel 168 expands to a fully expanded circumference, as shown in FIGS. 3A-3F. For example, while FIG. 3F shows the barrels 168 in a fully expanded circumference, in the crimped delivery state, the circumference of the barrel 168 may be crimped and not form a smooth circular/oval circumference as in the expanded configuration. The longitudinal struts 181-188 may be considered axial beams of the shunt structure 100; such struts, unlike the circumferential/lateral struts 120, can advantageously be straight. The S-curve shape/form of the lateral/circumferential struts 120 can include an inflection point 309 (see FIG. 3C). For a given strut 120, the inflection feature 309 can allow for the strut to curve with a convex curve facing both connected longitudinal struts.

Among the lateral/circumferential struts 120, the shunt structure 100 may include one or more apically-unsupported wishbone struts 124. The wishbone struts 124 may be coupled, for example, between the secondary pads 166 associated with the proximal arms 155 and the sensor holder 162. The wishbone struts 124 may comprise paired lateral struts that join at an apex 126, wherein the apex joint 126 is not coupled to a longitudinal strut. For example, whereas other joint connections of lateral/circumferential struts may be joined to a respective longitudinal strut, which may provide desirable mechanical stability for such joints, the wishbone joints 126 may not include such connections. Therefore, the structural integrity of the wishbone struts 124 may not be as strong as with other lateral/circumferential struts. However, the wishbone struts 124 may have greater flexibility and/or be malleable and conforming with respect to a shape thereof compared to certain other lateral/circumferential struts 120 of the shunt structure 100.

The shunt structure 100 may further include one or more partial wishbone struts, which include apex joints 129 that are connected on one axial side thereof to a longitudinal strut 183, but not on the other axial side. For example, the apex 129 may point in an axial direction associated with a side of the joint 129 that is connected to the longitudinal strut 183. Any of the lateral/circumferential struts 120 may be configured as a wishbone strut, or partial wishbone strut, or portion thereof. The joints 126, 127, 129 can generally form a U-shaped crown structure, or crown portion. Such crown structures can include a horizontal/lateral portion extending between and connecting the adjacent ends of the strut portions on either side, such that a gap is defined between the adjacent struts/strut portions and the crown structure connects the adjacent struts/portions at a location offset from the strut's natural point of intersection. The U-shaped crown structure(s) can reduce residual strains on the struts during crimping and expanding of the shunt 100 at the joints 126. 127, 129.

The barrel 168 can be configured as an annular, stent-like structure having a plurality of angularly spaced, vertically extending, axial beam struts (e.g., 181-188; collectively identified by reference 189). Axial struts 189 can be interconnected via a lower row R₁, an intermediate row R₂, and an upper row R₃ of angled (with respect to the axis A₁ of the barrel 168) and circumferentially-/laterally-extending struts 120. At least some of the struts 120 (e.g., the wishbone struts 124, partial wishbone struts 128, and other lateral struts that join on opposite sides of an axial beam strut (e.g., 184, 185) with mirrored/opposite deflection angles to collectively form a ‘wishbone’ shape) in each row advantageously are arranged in a zigzag or generally saw-tooth like pattern extending in the direction of the circumference of the frame 100, as shown.

The shunt frame 100 can be considered to have eight columns, wherein each column is defined by the struts/area extending laterally between each pair of adjacent axial beam struts 189. Alternatively, the number of columns may be considered to be the number of lateral segments between the secondary tissue contact pads 166 and that first distal arm 154a, as well as between the secondary tissue contact pads 166 and the sensor holder 162, totaling four columns C₁₋₄ in the illustrated embodiments. The number of columns and rows can be selected to reduce the overall crimp profile of the shunt. At least some of the wishbone and/or partial-wishbone struts of the first row R₂ may face in the opposite direction of at least some of the wishbone and/or partial-wishbone struts of the third row R₄ and/or second row R₂. Furthermore, at least some of the wishbone and/or partial-wishbone struts of the columns C₃₋₄ between the secondary contact pads 166 and the first distal anchor arm 154a may face in the opposite direction of at least some of the wishbone and/or partial-wishbone struts of the columns C₁₋₂ between the secondary contact pads 166 and the sensor holder 162.

The wishbone struts 124, 128 can be formed of adjacent strut portions in the same row that are interconnected to form an angle θ₉ between about 90 and 110 degrees, with about 100 degrees being a specific example. The selection of angle θ₉ between approximately 60-100°, such as between about 70-80°, such as about 75°, wherein such angles may optimize the radial strength of the barrel 168 when expanded, while still permitting the barrel 168 to be evenly crimped and expanded in the manner described herein.

Suitable plastically-expandable materials that can be used to form the shunt frame 100 and/or struts thereof include, without limitation, stainless steel, a nickel-based alloy (e.g., a nickel-cobalt-chromium alloy), polymers, or combinations thereof. In particular embodiments, frame 20 is made of a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight. It has been found that the use of MP35N to form shunt 100 provides superior structural results over stainless steel. In particular, when MP35N is used as the frame material, less material may be needed to achieve the same or better performance in radial and crush force resistance, fatigue resistances, and corrosion resistance. Moreover, since less material is required, the crimped profile of the frame can be reduced, thereby providing a lower profile valve assembly for percutaneous delivery to the treatment location in the body.

In some embodiments, one or more of the anchor arms of the shunt 100 may have certain covering associated therewith, such as the illustrated sock-like coverings 175 on the long arms 154. Such sock coverings 175 may be implemented over one or more surfaces or areas of the anchor arms 154 at or near a tissue contact area 164 of such arms. In some embodiments, the covers 175 comprise cloth sleeves/socks configured to promote tissue ingrowth from the tissue wall contact up the distal arms 154. Although coverings 175 are shown only on the distal long arms 154, it should be understood that such coverings may be implemented on the proximal arms 155 as well in some implementations.

The diameter and/or width of the barrel 168 may be between 5-10 mm such as about 7.5 mm. The various anchor arms 154, 155, as seen best from above in FIG. 3F, may have a somewhat triangular plan view shape, at least at a base portion thereof, which may be relatively wide at the junction with the barrel 168, wherein the anchor arms narrow to an apex at the terminal ends 161, 164. The elongated shape of the shunt 100 permits it to collapse down to a more linear profile so as to fit within a relatively small catheter. The anchor arms 154. 155, in the expanded configuration, can be curved in configuration, which also facilitates their collapse and expansion. That is, the struts that form the arms 154, 155 are designed so that they easily collapse into a compact size that fits into a delivery system/catheter when acted on by actuating rods or other actuating means, or through shape memory movement.

As mentioned above, the barrel 168 is defined by a generally parallel arrangement of curved chevron struts that forms an array of curved diamond-and chevron-shaped cells or openings 190. In some embodiments, the barrel 168 comprises two rows of cells 190 each stacked along the central axis A₁ that are offset lengthwise from each other. In some embodiments, the barrel/conduit form/body 168 that defines the shunt orifice may be covered internally and/or externally, at least in part, with fabric or other covering, which may provide sealing for the device.

The length of the proximal anchor features 155 may be considered the length/diameter L₃ of the contact pad 161 or the length/dimension L₅ of the contact pad 161 and the longitudinal strut 185. The length of the distal anchor arm 154 may be considered the length L₁ of the contact pad 164 and the elongate strut/arm 163, or may be considered the length Lo that includes the axial/vertical strut 183, or may be considered the length L₇ comprising the dimension of the pad 164, strut/arm 163, and axial length of the side strut(s) 123 that support the arm/strut 163 on either side. Regardless of how the length of the distal arms 154 are considered, such length can advantageously be greater than the length of the proximal anchor features/arms 155. The length of the barrel 168 can be considered as the illustrated length L₂.

The dimensions L₁-L₇ can have any suitable or desirable values. In some embodiments, the dimension L₁ has a value of between 5-15 mm, such as between 10-12 mm. For example, L₁ can have a value of about 10.3 mm. In some embodiments, the dimension L₂ has a value of between 4-8 mm, such as between 5-7 mm. For example, L₂ can have a value between 5.9-6 mm (e.g., about 5.93 mm). In some embodiments, the dimension L₃ has a value of between 1-3 mm, such as between 1.5-2.3 mm. For example, L₃ can have a value of between 1.9-2 mm (e.g., about 1.9 mm). In some embodiments, the dimension L₄ has a value of between 2.7-4.7 mm, such as between 3.2-4.2 mm. For example, L₄ can have a value of between 3.7-3.8 mm (e.g., about 3.72 mm). In some embodiments, the dimension L₅ has a value of between 4-8 mm, such as between 5-7 mm. For example, L₅ can have a value between 5.9-6 mm (e.g., about 5.93 mm). In some embodiments, the dimension L₆ has a value of between 10-14 mm, such as between 11-13 mm. For example, L₆ can have a value of between 11.7-12 mm (e.g., about 11.8 mm). In some embodiments, the dimension L₇ has a value of between 12-15 mm, such as between 13-14 mm. For example, L₇ can have a value of between 13.4-13.5 mm (e.g., about 13.45 mm). Although particular dimensional values are listed above, it should be understood that the values of the various dimensions may be any value between the listed ranges, or values outside of such ranges with respect to one or more of the illustrated dimensions.

FIGS. 4A, 4B, and 4C show perspective, exploded perspective, and axial views, of a shunt implant device 100 having a sensor 60 associated therewith in accordance with one or more embodiments. The shunt implant device 100 shown in FIGS. 4A-4C can be the same or similar as the shunt implant device shown in FIGS. 3A-3G and described in detail above. As referenced above, shunt and/or other implant devices/structures may be integrated with sensor, antenna/transceiver, and/or other components to facilitate in vivo monitoring of pressure and/or other physiological parameter(s). Sensor devices in accordance with embodiments of the present disclosure may be integrated with cardiac shunt structures/devices or other implant devices using any suitable or desirable attachment or integration mechanism or configuration.

In some embodiments, the sensor device/assembly 60 includes a sensor transducer component 65 and an antenna component, which may be disposed within a housing 69 of the sensor device 60. The sensor transducer component 65 may comprise any type of sensor transducer as described in detail above. In some embodiments, the sensor device 60 may be attached to or integrated with a sensor holder 162 of the shunt structure 100, as shown. For example, the sensor holder 162 may be generally associated with a proximal (or distal) axial portion/end of the shunt structure 100. That is, when the shunt structure 100 is implanted, a distal end of the barrel 168 may be associated with an inlet opening/portion of the shunt structure 100, whereas proximal end of the barrel 168 may be associated with an outlet opening/portion of the shunt structure 100. Although distal and proximal sides/portions are described in some contexts herein, it should be understood that identified distal portions/sides may be outlet or inlet sides of the relevant shunt structure, as with identified proximal portions/sides. Furthermore, the terms “distal” and “proximal” are used for convenience and may or may not refer to relative orientation with respect to a delivery system/device used to implant the relevant sensor implant device and/or shunt structure.

The sensor transducer component 65 includes a sensor clement 67, such as a pressure sensor transducer/membrane. Relative to the sensor holder 162 of the shunt structure 100, the sensor device 60 may be attached/positioned at/on a proximal portion 62 thereof to the sensor holder 162, or any other portion of the housing 69 or sensor 60. In some embodiments, readings acquired by the sensor device 60 may be used to guide titration of medication for treatment of a patient in whom the implant device 100 is implanted.

As described herein, the sensor device 60 may be configured to implement wireless data and/or power transmission. The sensor device 60 may include the antenna component for such purpose. The antenna(s), as well as one or more other components of the sensor device 60, may be contained at least partially within the sensor housing 69, which may further have disposed therein certain control circuitry configured to facilitate wireless data and/or power communication functionality. In some embodiments, the antenna(s) may comprise one or more conductive coils, which may facilitate inductive powering and/or data transmission. In embodiments comprising conductive coil(s), such coil(s) may be wrapped/disposed at least partially around a magnetic (e.g., ferrite, iron) core.

The sensor device 60 may be attached to, or integrated with, the shunt structure 100 in any suitable or desirable way. For example, in some implementations, the sensor device 60 may be attached or integrated with the shunt structure 100 using mechanical attachment means (e.g., sensor holder 162 and/or attachment means 61). In some embodiments, the sensor device 60 may be contained in a pouch or other receptacle that is attached to the shunt structure 100. The sensor holder 162 can be configured to deflect/fold outwardly from the axis of the barrel and act as a stopper feature/tab to cover at least a portion of the radial profile of the sensor device 60 when the sensor device is attached thereto in a manner as to restrict axial movement and/or angular deflection of the sensor device 60 in at least one direction. In some embodiments, the sensor device 60 is integrated with the sensor holder 162, such that separate attachment means/feature(s) are not necessary to secure the sensor device 60 to the shunt structure 100. For example, the sensor holder/arm 162 may be integral with the housing 69 of the sensor device 60.

In some embodiments, the sensor holder 162 includes a ring or other form having an aperture therein, wherein the proximal base 62 of the sensor housing 69 can be configured to be placed on the sensor holder 162, wherein a screw or other fixation means 61 is coupled to the base at least partially through the aperture 191 of the sensor holder 162. For example, the attachment means (e.g., screw) 61 can include a male or female threading that is configured to mate with corresponding female or male threading associated with the base 62 of the sensor housing 69. The attachment means 61 may include a proximal flange 192 having a diameter that is wider in at least one or more portions thereof than the corresponding diameter of the aperture 191 of the sensor holder 162. Therefore, when the attachment projection 193 of the attachment means 61 (e.g., screw, clip, clamp, etc.) is extended through the aperture 191 and engaged with the base 62 of the sensor housing 69, the flange 192 of the attachment means 61 can sandwich the ring form/structure of the sensor holder 162 between the flange 192 and the base 62 of the sensor housing 69, wherein the base 62 of the sensor housing is likewise wider than the aperture 191 in one or more areas, thereby preventing the sensor housing 69 from passing through the aperture 191. The attachment means 61 may advantageously be screwed or attached relatively tightly against the sensor holder 162, to thereby prevent undesirable movement or dislodging/unwinding of the sensor housing 69 therefrom. In some implementations, the attachment means 61 may be locked, glued, or otherwise permanently fixed to the base 62 of the housing 69 and/or the sensor holder 162.

Although the attachment means 61 is shown as having a male screw projection 193, wherein the base 62 of the sensor housing 69 comprises a corresponding female screw recess, in some implementations, the male screw projection may be associated with the base 62 of the housing 69, whereas the attachment means 61 may include a female screw recess configured to receive the screw projection associated with the base 62 of the housing. Although the attachment means 61 is shown and described as a screw-type device, it should be understood that the securement/attachment means/mechanism implemented for attaching the sensor device 60 to any of the arms of the shunt structure 90 may be any of the features disclosed in PCT Application No. PCT/US20/56746, Filed on Oct. 22, 2020, and entitled “Sensor Integration in Cardiac Implant Devices,” the contents of which are hereby expressly incorporated by reference in their entirety. For example, the shunt structure 100 and/or arm(s) thereof may include one or more sensor-retention fingers, clamps, wraps, bands, belts, clips, pouches, housings, encasements, and/or or other attachment means configured to secure the sensor device 60 to an arm, strut, or other holder structural feature of the shunt structure 100.

The sensor holder 162 may be associated with either axial side/end of the shunt structure 100, wherein the different axial sides/ends of the shunt structure 100 are exposed on opposite sides of a tissue wall when the implant device 100 is implanted in the tissue wall. As described herein, references to axial sides of a shunt structure may refer to opposite sides of a plane P, axially (and/or diagonally, as in FIG. 3C) bisecting the shunt structure 100 and/or barrel portion 168 thereof.

The sensor device 60 may advantageously be biocompatible. For example, the housing 69 may advantageously be biocompatible, such as a housing comprising glass or other biocompatible material. However, at least a portion of the sensor transducer element/membrane 67, such as a diaphragm or other component, may be exposed to the external environment in some embodiments in order to allow for pressure readings, or other parameter sensing, to be implemented. The housing 69 may comprise an at least partially rigid cylindrical or tube-like form, such as a glass cylinder form. In some embodiments, the sensor transducer component 65/67 is approximately 3 mm or less in diameter. The antenna contained within (or without) the housing 69 may be approximately 20 mm or less in length.

The sensor device 60 may be configured to communicate with an external system when implanted in a heart or other area of a patient's body. For example, the antenna(s) may receive power wirelessly from the external system and/or communicate sensed data or waveforms to and/or from the external system.

The sensor element 67 may comprise a pressure transducer. For example, the pressure transducer may be a microelectromechanical system (MEMS) transducer comprising a semiconductor diaphragm component. In some embodiments, the transducer may include an at least partially flexible or compressible diaphragm component, which may be made from silicone or other flexible material. The diaphragm component may be configured to be flexed or compressed in response to changes in environmental pressure. The control circuitry contained within the housing 69 may be configured to process signals generated in response to said flexing/compression to provide pressure readings. In some embodiments, the diaphragm component is associated with a biocompatible layer on the outside surface thereof, such as silicon nitride (e.g., doped silicon nitride) or the like. The diaphragm component and/or other components of the pressure transducer 67 may advantageously be fused or otherwise sealed to/with the housing 69 of the sensor device 60 in order to provide hermetic sealing of at least some of the sensor components.

The control circuitry implemented in (and/or electrically and/or physically coupled to) the sensor device 60 may comprise one or more electronic application-specific integrated circuit (ASIC) chips or die, which may be programmed and/or customized or configured to perform monitoring functionality as described herein and/or facilitate transmission of sensor signals wirelessly. The antenna(s) may comprise a ferrite core wrapped with conductive material in the form of a plurality of coils (e.g., wire coils). In some embodiments, the coils comprise copper or other metal. The antenna(s) may advantageously be configured with coil geometry that does not result in substantial displacement or heating in the presence of magnetic resonance imaging. In some implementations, the sensor implant device 100 may be delivered to a target implant site using a delivery catheter (not shown), wherein the delivery catheter includes a cavity, recess, channel, or the like configured to accommodate the advancement of the sensor device 60 therethrough.

The sensor holder 162 may emanate radially outward from the barrel 168, wherein, in a deployment configuration as shown in FIGS. 4A-4C, the sensor holder 162, as with the arms and/or other anchoring features of the shunt structure 100, extends radially outward from the fluid conduit 168. Conversely, in a delivery configuration, as described in greater detail below, the sensor holder 162 may extend generally axially with respect to the barrel/conduit axis.

With the sensor device 60 secured to the shunt structure 100 as shown in FIGS. 4A-4C, at least a portion of the sensor device 60 may be situated outside of, and/or parallel to, the barrel 168, such that when the shunt implant device 100 is implanted in an opening in a tissue wall, at least a portion of the sensor device 60 is disposed between at least a portion of the barrel portion 168 of the shunt implant 100 and an inner surface/perimeter of the opening (e.g., hole) in the tissue wall. For example, the sensor device 60 may be considered to be sandwiched between the barrel 168 and the tissue wall/opening in which the shunt implant 100 is implanted. With the sensor device 60 situated between the inside surface of the opening in the tissue wall and the barrel 168 of the shunt implant 100, the barrel 168 may be configured to establish only a partial circle of contact with the inside surface of the opening. For example, the sensor device 60 may be in contact with a circumferential portion/segment of the barrel 168 and/or may prevent the circumferential portion/segment of the barrel that is aligned with the sensor housing 69 from contacting the inside surface of the opening in the tissue wall. Instead, the sensor device 60 may be in contact with the inside surface of the opening and/or the barrel 168.

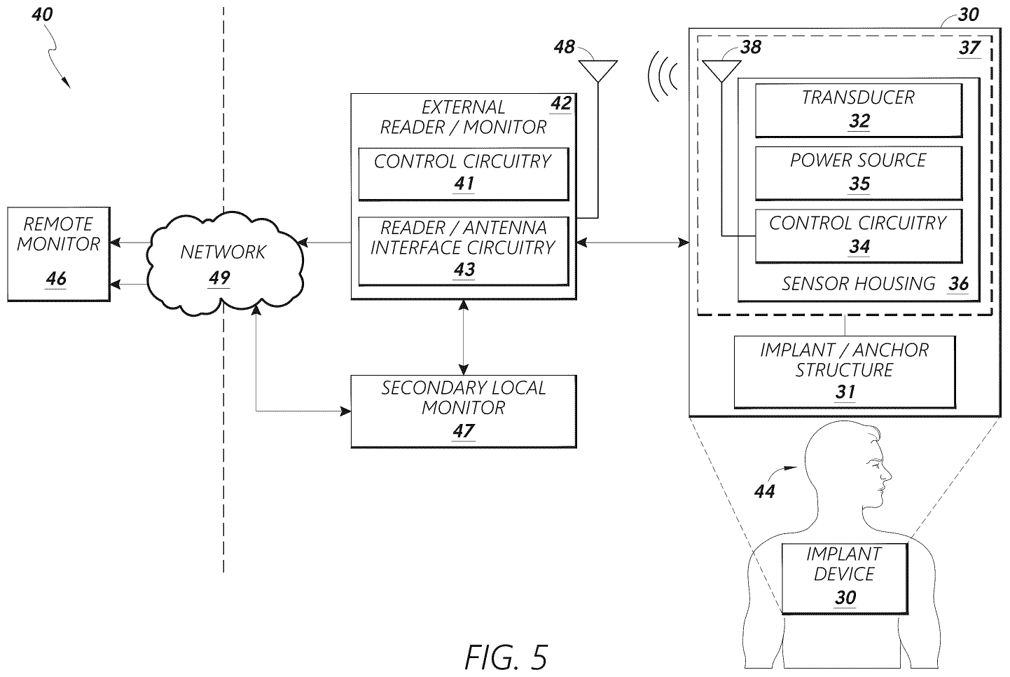

FIG. 5 shows a system 40 for monitoring one or more physiological parameters (e.g., left atrial pressure and/or volume) in a patient 44 according to one or more embodiments. The patient 44 can have a medical implant device 30 implanted in, for example, the heart (not shown), or associated physiology, of the patient 44. For example, the implant device 30 can be implanted at least partially within the left atrium and/or coronary sinus of the patient's heart. The implant device 30 may be an embodiment of any of the shunt implant devices disclosed herein, wherein such devices are associated with a sensor device. The implant device 30 can include one or more sensor transducers 32, such as one or more microelectromechanical system (MEMS) devices (e.g., MEMS pressure sensors, or other type of sensor transducer).

In certain embodiments, the monitoring system 40 can comprise at least two subsystems, including an implantable internal subsystem or device 30 that includes the sensor transducer(s) 32, as well as control circuitry 34 comprising one or more microcontroller(s), discrete electronic component(s), and one or more power and/or data transmitter(s) 38 (e.g., antennae coil). The monitoring system 40 can further include an external (e.g., non-implantable) subsystem that includes an external reader 42 (e.g., coil), which may include a wireless transceiver that is electrically and/or communicatively coupled to certain control circuitry 41. In certain embodiments, both the internal 30 and external 42 subsystems include one or more corresponding coil antennas for wireless communication and/or power delivery through patient tissue disposed therebetween. The sensor implant device 30 can be any type of implant device. For example, in some embodiments, the implant device 30 comprises a pressure sensor integrated with another functional implant structure 31, such as a prosthetic shunt or stent device/structure.

Certain details of the implant device 30 are illustrated in the enlarged block 30 shown. The implant device 30 can comprise an implant/anchor structure 31 as described herein. For example, the implant/anchor structure 31 can include a percutaneously-deliverable shunt device configured to be secured to and/or in a tissue wall to provide a flow path between two chambers and/or vessels of the heart, as described in detail throughout the present disclosure.

The implant/anchor structure 31 can include one or more anchoring arms or features associated with each axial end of the implant device and/or barrel thereof. Furthermore, as referenced above, shunt implant devices disclosed herein can include chevron-type circumferential struts. The use of chevron struts with shunt implant devices as disclosed herein can provide improved collapsibility of the shunt barrel. Furthermore, implementation of offset axial arms, wherein one or more anchor arms associated with a first side of the implant device is/are offset relative to one or more (or all) anchor arms associated with a second side of the implant device, can facilitate improved anchoring and/or allow for decreased transportation/compressed profile.

Although certain components are illustrated in FIG. 5 as part of the implant device 30, it should be understood that the sensor implant device 30 may only comprise a subset of the illustrated components/modules and can comprise additional components/modules not illustrated. The implant device may represent an embodiment of the implant device shown in FIGS. 3A-3G and/or 4A-4C, and vice versa. The implant device 30 can advantageously include one or more sensor transducers 32, which can be configured to provide a response indicative of one or more physiological parameters of the patient 44, such as atrial pressure. Although pressure transducers are described, the sensor transducer(s) 32 can comprise any suitable or desirable types of sensor transducer(s) for providing signals relating to physiological parameters or conditions associated with the implant device 30 and/or patient 44.

The sensor transducer(s) 32 can comprise one or more MEMS sensors, optical sensors, piezoelectric sensors, electromagnetic sensors, strain sensors/gauges, accelerometers, gyroscopes, diaphragm-based sensors, and/or other types of sensors, which can be positioned in the patient 44 to sense one or more parameters relevant to the health of the patient. The transducer 32 may be a force-collector-type pressure sensor. In some embodiments, the transducer 32 comprises a diaphragm, piston, bourdon tube, bellows, or other strain-or deflection-measuring component(s) to measure strain or deflection applied over an area/surface thereof. The transducer 32 may be associated with the sensor housing 36, such that at least a portion thereof is contained within, or attached to, the housing 36.

In some embodiments, the transducer 32 comprises or is a component of a strain gauge, which may be configured to use a bonded or formed strain gauge to detect strain due to applied pressure. For example, the transducer 32 may comprise or be a component of a piezoresistive strain gauge, wherein resistance increases as pressure deforms the component/material of the strain gauge. The transducer 32 may incorporate any type of material, including but not limited to silicone, polymer, silicon (e.g., monocrystalline), polysilicon thin film, bonded metal foil, thick film, silicon-on-sapphire, sputtered thin film, and/or the like. In some embodiments, a metal strain gauge is adhered to the sensor surface, or a thin-film gauge may be applied on the sensor by sputtering or other technique. The measuring element or mechanism may comprise a diaphragm or metal foil. The transducer 32 may comprise any other type of sensor or pressure sensor, such as optical, potentiometric, resonant, thermal, ionization, or other types of strain or pressure sensors.

In some embodiments, the transducer 32 comprises or is a component of a capacitive pressure sensor including a diaphragm and pressure cavity configured to form a variable capacitor to detect strain due to pressure applied to the diaphragm. The capacitance of the capacitive pressure sensor may generally decrease as pressure deforms the diaphragm. The diaphragm may comprise any material(s), including but not limited to metal, ceramic, silicone, silicon or other semiconductor, and the like. In some embodiments, the transducer 32 comprises or is a component of an electromagnetic pressure sensor, which may be configured to measures the displacement of a diaphragm by means of changes in inductance, linear variable displacement transducer (LVDT) functionality, Hall Effect, or eddy current sensing. In some embodiments, the transducer 32 comprises or is a component of a piezoelectric strain sensor. For example, such a sensor may determine strain (e.g., pressure) on a sensing mechanism based on the piezoelectric effect in certain materials, such as quartz.

In some embodiments, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In certain embodiments, the sensor transducer(s) 32 can be configured to generate electrical signals that can be wirelessly transmitted to a device outside the patient's body, such as the illustrated local external monitor system 42. In order to perform such wireless data transmission, the implant device 30 can include radio frequency (RF) (or other frequency band) transmission circuitry, such as signal processing circuitry and an antenna 38. The antenna 38 can comprise an antenna coil implanted within the patient. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some embodiments.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions from the implant device 30 and/or provide wireless power to the implant device 30 using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain embodiments, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain embodiments, the local monitor 42 comprises a smartphone, laptop computer, or other mobile computing device, or any other type of computing device.

In certain embodiments, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or other external subsystem. In certain embodiments, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain embodiments, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. In certain embodiments, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5. 10, 20, 30, 40, or 90 days, or other period of time).

In some embodiments, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain embodiments, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. In an embodiment, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47. and/or implant device 30. For example, the remote monitor subsystem 46 can advantageously be operated and/or controlled by a healthcare entity, such as a hospital, doctor, or other care entity associated with the patient 44. Although certain embodiments disclosed herein describe communication with the remote monitor subsystem 46 from the implant device indirectly through the local monitor device 42, in certain embodiments, the implant device 30 can comprise a transmitter capable of communicating over the network 49 with the remote monitor subsystem 46 without the necessity of relaying information through the local monitor device 42.

In some embodiments, at least a portion of the transducer 32, control circuitry 34, power source 35 and/or the antenna 38 are at least partially disposed or contained within the sensor housing 36, which may comprise any type of material, and may advantageously be at least partially hermetically sealed. For example, the housing 36 may comprise glass or other rigid material in some embodiments, which may provide mechanical stability and/or protection for the components housed therein. In some embodiments, the housing 36 is at least partially flexible. For example, the housing may comprise polymer or other flexible structure/material, which may advantageously allow for folding, bending, or collapsing of the sensor 37 to allow for transportation thereof through a catheter or other percutaneous introducing means.

FIG. 6 shows a shunt implant device 100 implanted in a coronary sinus tissue wall 21 in accordance with one or more embodiments. As referenced above, in some implementations, shunt devices/structures in accordance with embodiments of the present disclosure may be implanted in a wall 21 separating the coronary sinus 16 from the left atrium 2, such that interatrial shunting may be achieved through the coronary sinus 16. FIG. 6 shows a section of the heart from a top-down, superior perspective with the posterior aspect oriented at the top of the page. The implant/anchor structure of the implant device 100 can comprise a shunt structure having axially and/or circumferentially offset anchor arms/pads in some embodiments, as disclosed in detail herein. Furthermore, the implant/anchor structure may comprise chevron/zigzag lateral/circumferential struts, as described in detail herein.

In some cases, left-to-right shunting through implantation of the shunt device 100 in the wall 21 between the left atrium 2 and the coronary sinus 16 can be preferable to shunting through the atrial septum 18. For example, shunting through the coronary sinus 16 can provide reduced risk of thrombus and embolism. Generally, the coronary sinus can be less likely to have thrombus/emboli present for several reasons. First, the blood draining from the coronary vasculature into the right atrium 5 has just passed through capillaries, so it is essentially filtered blood. Second, the ostium 14 of the coronary sinus in the right atrium is often partially covered by a pseudo-valve called the Thebesian valve (not shown). The Thebesian valve is not always present, but, where present, it can block thrombus or other emboli from entering in the event of a spike in right atrial pressure. Third, the pressure gradient between the coronary sinus and the right atrium into which it drains is generally relatively low, such that thrombus or other emboli in the right atrium is likely to remain there. Fourth, in the event that thrombus/emboli do enter the coronary sinus, there is typically a much greater gradient between the right atrium and the coronary vasculature than between the right atrium and the left atrium. Most likely, thrombus/emboli would travel further down the coronary vasculature until right atrium pressure returned to normal and then the emboli would return directly to the right atrium.

Some additional advantages to locating the shunt implant device 100 in the wall 21 between the left atrium 2 and the coronary sinus 16 relate to the consideration that such anatomy is generally more stable than the interatrial septal tissue. By diverting left atrial blood into the coronary sinus, sinus pressures may increase by a small amount. This can cause blood in the coronary vasculature to travel more slowly through the heart, possibly increasing perfusion and oxygen transfer, which can be more efficient and also can help a dying heart muscle to recover. In addition, by implanting the shunt device/structure 100 in the wall of the coronary sinus, damage to the atrial septum 18 may be prevented. Therefore, the atrial septum 18 may be preserved for later transseptal access for other therapies. The preservation of transseptal access may be advantageous for various reasons. For example, heart failure patients often have a number of other comorbidities, such as atrial fibrillation and/or mitral regurgitation; certain therapies for treating these conditions require a transseptal access.

It should be noted, that in addition to the various benefits of placing the sensor implant device 100 between the coronary sinus 16 and the left atrium 2, certain drawbacks may be considered. For example, by shunting blood from the left atrium 2 to the coronary sinus 16. oxygenated blood from the left atrium 2 may be passed to the right atrium 5 and/or non-oxygenated blood from the right atrium 5 may be passed to the left atrium 2, both of which may be undesirable with respect to proper functioning of the heart.

With further reference to FIG. 6, the coronary sinus 16 is generally contiguous around the left atrium 2, and therefore there are a variety of possible acceptable placements for the implant device 100. The target site selected for placement of the implant device 100 may be made in an area where the tissue of the particular patient is less thick or less dense, as determined beforehand by non-invasive diagnostic means, such as a CT scan or radiographic technique, such as fluoroscopy or intravascular coronary echo (IVUS).

As with other embodiments, the shunt implant device 100 can include a sensor device 60 having a sensor transducer component 65 and certain connectivity component(s) (e.g., an antenna component and/or other control circuitry). The shunt implant device 100 is disposed, attached, and/or otherwise secured to or associated with one or more anchor arms associated with and or coupled to a barrel structure 168 of the shunt structure 100 in a manner such that the sensor transducer 65 is disposed the left atrium 2, as shown, or alternatively (or additionally) in the coronary sinus 16. For example, the implant device 100 may be configured such that the sensor transducer component 65 is at least partially exposed on the atrial side of the tissue wall 21, as shown. The sensor device 60 may be configured to be held/secured on an outside of the barrel 168, such that the sensor housing is positioned/pinned between the barrel 168 and the tissue wall 21 inside the opening in which the barrel 168 is deployed/implanted. With the sensor transducer component 65 disposed in an area outside and/or near the barrel inlet, the sensor transducer 65 may advantageously be disposed in an area of flow that is relatively high, thereby allowing for sensor readings to be generated indicating characteristics of the flow through the barrel 168 of the shunt structure 100.

FIG. 7 shows a shunt implant device 100 implanted in an atrial septum 18 in accordance with one or more embodiments. FIG. 7 shows a sensor device 60 associated with the shunt implant 100, as described herein, wherein the sensor 60 of the device 100 is exposed in the left atrium 2 in accordance with one or more embodiments. As with other embodiments, the sensor device 60 includes a sensor transducer component 65 and a cylindrical housing. The sensor device is disposed, attached, and/or otherwise secured or to or associated with the shunt structure of the implant device 100 in a manner such that the sensor housing is positioned between the outside diameter of the barrel 168 of the shunt implant 100 and the perimeter of the opening in the tissue wall 18 in which the barrel 168 is disposed, which may advantageously provide relatively secure placement/fixation of the sensor device. Although FIG. 7 shows the implant device 100 implanted with the sensor transducer 65 disposed on the left atrial side of the wall 18, it should be understood that embodiments of the present disclosure can be implanted in an atrial (or ventricular) septum with the sensor transducer thereof disposed/exposed on the right side of the septum.

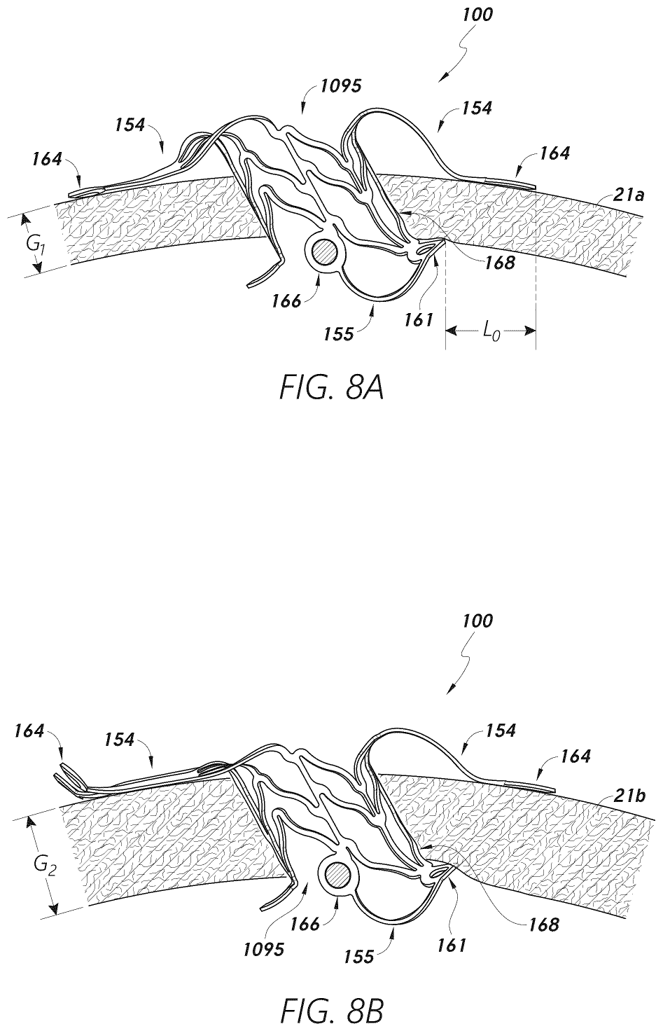

FIGS. 8A and 8B show a shunt implant device 100 implanted in tissue wall segments having varying thicknesses in accordance with one or more embodiments. With reference to both FIGS. 8A and 8B, when implanted, the distal anchor arms 154 of the implant device 100 curve/bulge axially away from the barrel 168 of the shunt 100 and deflect radially outward and back toward the axial center (as aligned with the plane of the tissue wall 21) of the barrel 168 moving towards the terminal ends thereof. The proximal arms 155 may deflect radially outward in a manner to impede axial movement of the shunt 100 through the opening 1095 in the tissue wall 21. Therefore, the radially-extended distal 154 and proximal 155 anchor arms can be configured to hold/pinch the tissue wall 21 therebetween, thereby securing the shunt implant 100 in place in the tissue wall 21.

The terminal ends/pads 164 of the distal anchor arms 154 are configured to contact and/or grip the tissue wall 21, thus helping to maintain the shunt 100 in place. The length and/or material characteristics of the distal anchor arms 164 can allow for the shunt 100 to be able to accommodate tissue walls of varying thicknesses. For example, tissue wall segments may vary in thickness across patients and across different tissue segments and anatomies in a patient. FIG. 8A shows the implant 100 implanted in a relatively thin tissue wall 21a, whereas FIG. 8B shows the implant 100 implanted in a relatively thicker tissue wall 21b, wherein the length and/or material characteristics of the long distal anchor arms 15 allow for the implant 100 to securely hold itself in place in either tissue wall.

Due to the increased length of the distal anchor arms 154 relative to the proximal anchor arms 155, the contact pads 164 of the distal arms 154 may generally contact the tissue wall 21 at a position that is laterally offset by an amount/distance Lo relative to the tissue contact position of the primary 161 (and/or secondary 166) contact pad(s) of the proximal arms 155. Such lateral offset can serve to disperse the clamping force of the anchor arms 154, 155 over an expanded area of the tissue wall 21, which can provide increased stability for the implant 100 and/or reduce direct pinching of the tissue wall 21 between the distal and proximal anchor arms.

In some embodiments, the shunt 100 and/or long distal anchor arms 154 may comprise super-elastic material, such as nitinol or other memory metal, which may cause the arms 154 to exhibit relatively high flexibility, which may allow for the shape memory of the arms to deflect the arms toward the tissue wall 21 without applying excessive clamping forces to the wall that could possibly cause necrosis or other tissue damage.

The length of the distal arms 154 can provide flexibility in the vertical/axial position of the contact pads 164, wherein different amounts and/or shapes/angles of bending/flexing in the arms 154 can result in different vertical/axial positions of the contact pads 164 relative to the axis of the shunt 100. For example, the distal anchor arms 154 may advantageously have a length L₁ (see FIG. 3G) that is greater than a length L₂ (see FIG. 3G) of the barrel 168. In some embodiments, the distal anchor arms 154 are between about 30-70% or 40-60% longer than the barrel 168, such as about 50% longer. Conversely, the proximal arms 155 may be shorter, even significantly shorter, than the barrel 168. In some embodiments, the length of the proximal arms 155 is attributable solely to the width/length of the contact pads 161. 166 and/or vertical offset arms 169.

Depending on the bend/flexed configuration of the distal anchor arms 154, the axial gap between the contact pads of the distal and proximal anchor arms may be varied to accommodate the thickness of a particular tissue wall. For example, in FIG. 8A, the anchor arms 154 may be deflected back toward the tissue wall 21a to a greater degree than in FIG. 8B, such that the gap G₁ between the distal and proximal anchor arms is less than the gap G₂ shown in FIG. 8B. That is, in the implementation of FIG. 8B, the tissue wall 21b is relatively thicker, and therefore, the distal anchor arms 154 may be inclined to deflect downward (with respect to the illustrated orientation) to a lesser degree than in FIG. 8A in order to contact and/or apply the desired pressure against the tissue wall 21b.

As shown in FIGS. 8A and 8B, when in the expanded/deployed configuration, the shunt 100 may have a tilted orientation with respect to the axis of the barrel 168 relative to the tissue wall 21. In such implementations, the shunt 100 can be implanted in the tissue wall 21 using a catheter that passes through the tissue wall at an angle. Once expanded, the anchor arms 154, 155 clamp about the tissue wall 21 and maintain the barrel within the puncture opening in the tissue wall. When deployed, in some embodiments, the long distal anchor arms 154 bend at an obtuse angle, while the proximal anchor arms 155 and/or associated contact pads 161 are bent/deflected at an acute angle, as shown.

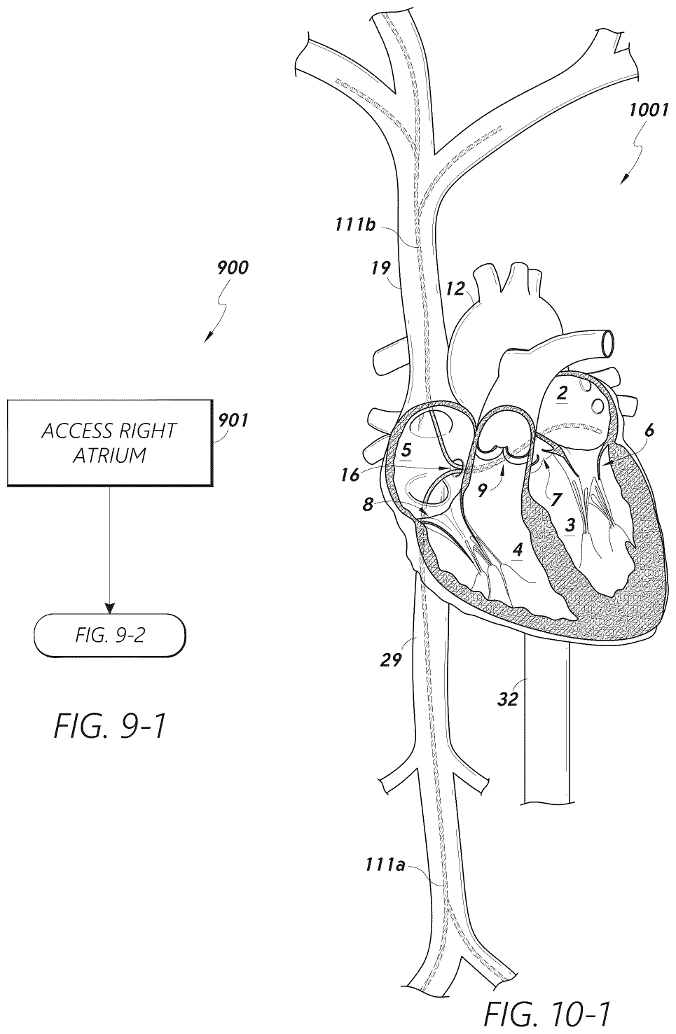

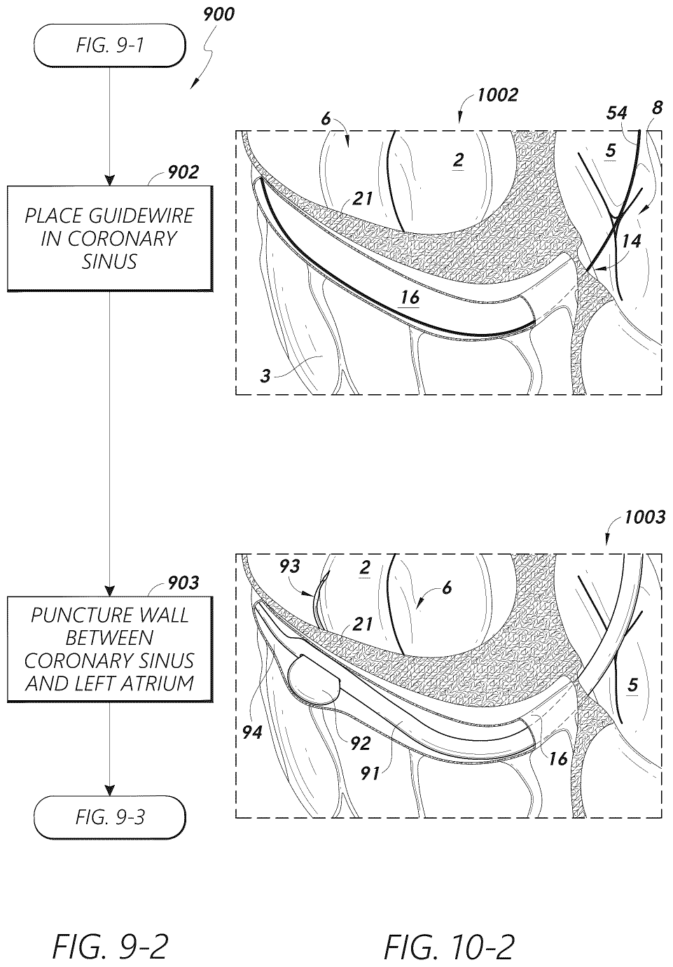

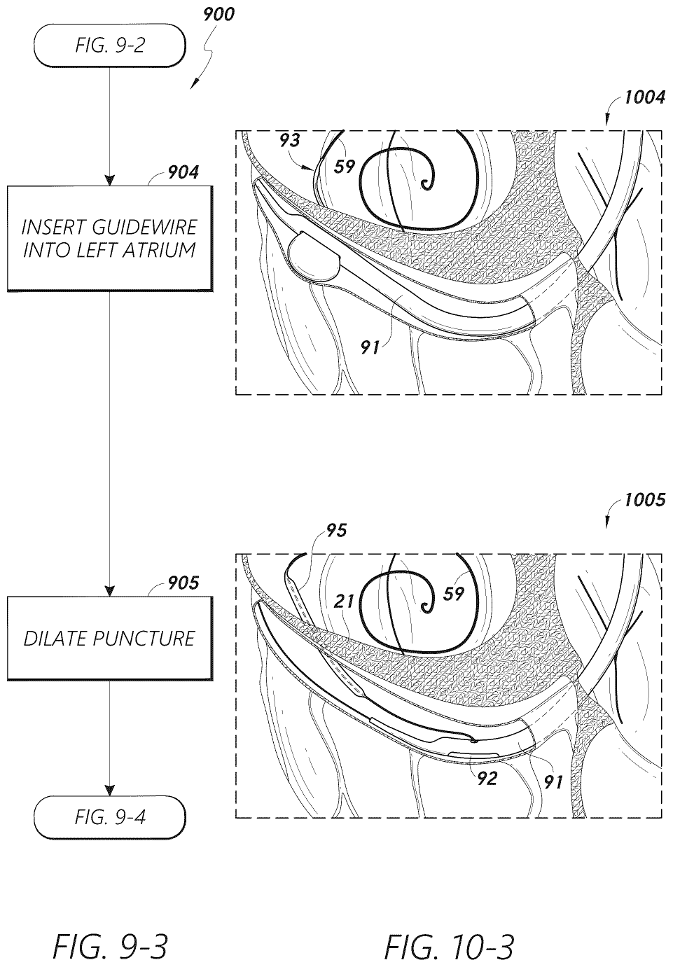

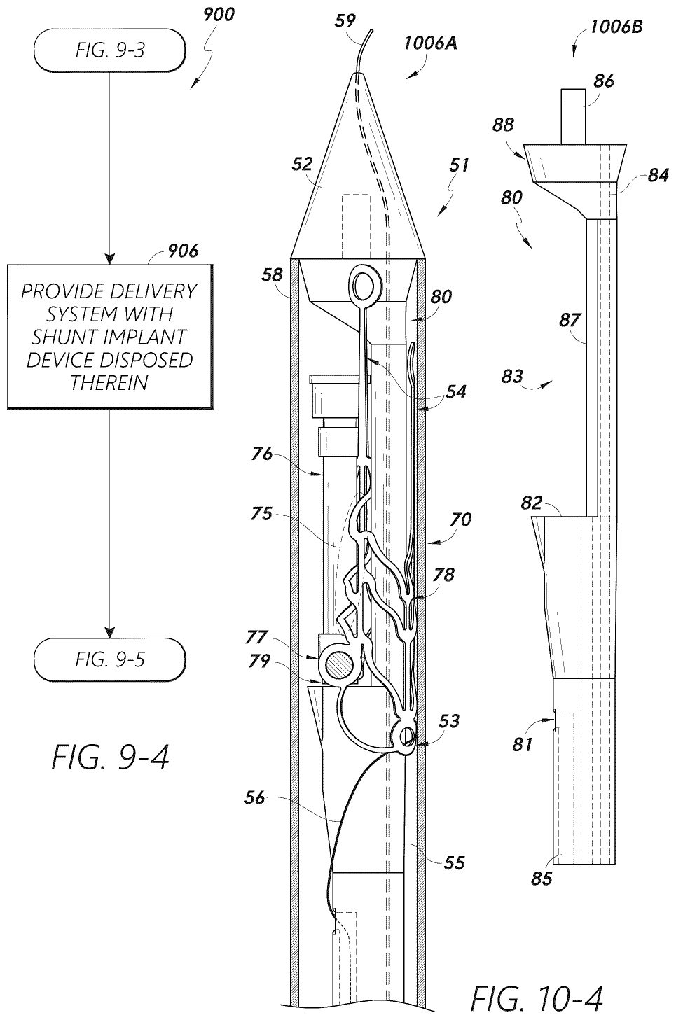

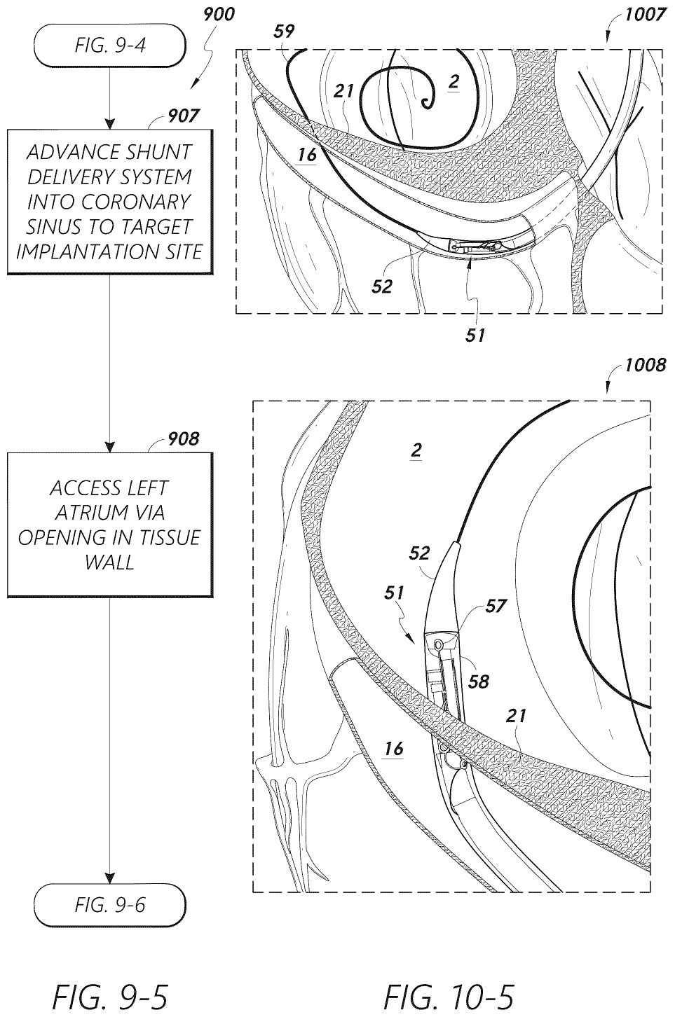

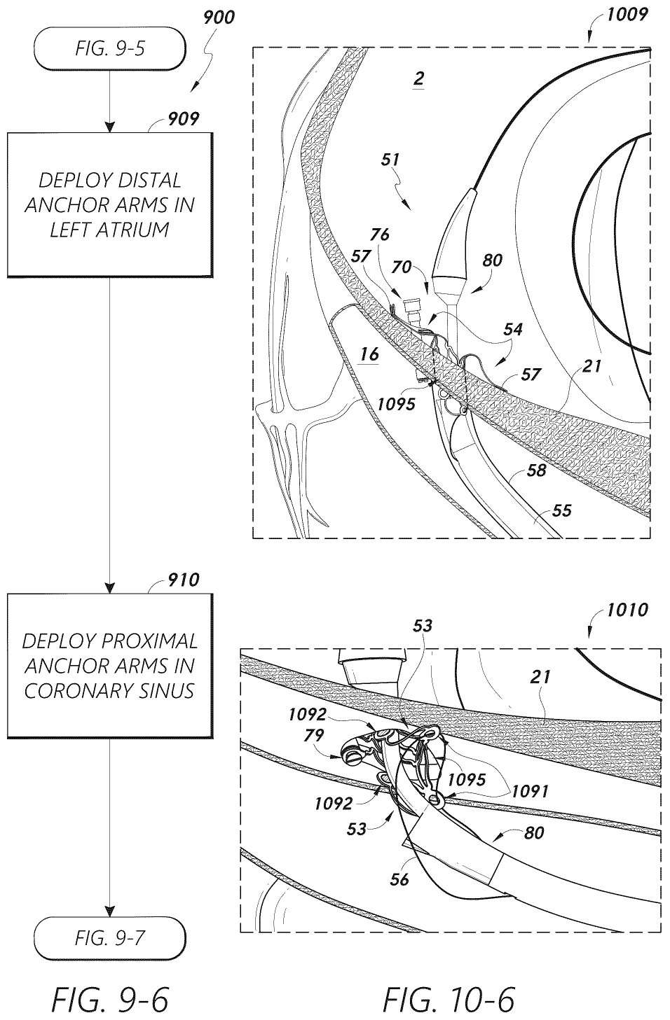

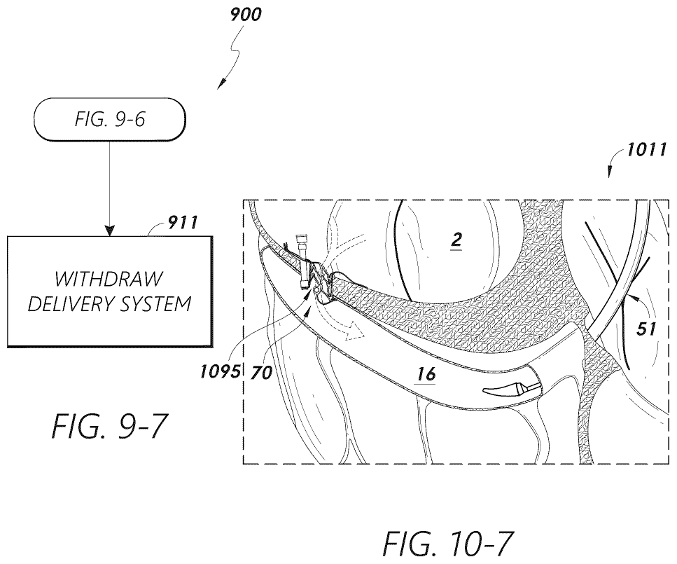

FIGS. 9-1, 9-2, 9-3, 9-4, 9-5, 9-6, and 9-7 provide a flow diagram illustrating a process 900 for implanting a shunt device in accordance with one or more embodiments. FIGS. 10-1, 10-2, 10-3, 10-4, 10-5, 10-6, and 10-7 provide images of cardiac anatomy and certain devices/systems corresponding to operations of the process 900 of FIGS. 9-1, 9-2, 9-3, 9-4, 9-5, 9-6, and 9-7 in accordance with one or more embodiments.

At block 901, the process 900 involves accessing a right atrium 5 of a heart of a patient with a catheter-based delivery system 111. Image 1001 shows various catheter-/sheath-type delivery systems 111 that may be used to implant shunt devices in accordance with aspects of the present disclosure. The delivery systems 111 can advantageously be steerable and relatively small in cross-sectional profile to allow for traversal of the various blood vessels and chambers through which they may be advanced en route to, for example, the right atrium 5, coronary sinus 16, left atrium 2 or other anatomy or chamber. Catheter access to the right atrium 5, coronary sinus 16, or left atrium 2 in accordance with certain transcatheter solutions may be made via the inferior vena cava 29 (as shown by the catheter 111a) or the superior vena cava 19 (as shown by the catheter 111b).

Although access to the right and/or left atria is illustrated and described in connection with certain examples as being via the right atrium and/or vena cavae, such as through a transfemoral or other transcatheter procedure, other access paths/methods may be implemented in accordance with examples of the present disclosure. For example, in cases in which septal crossing through the interatrial septal wall is not possible, other access routes may be taken to the left atrium 2. In patients suffering from a weakened and/or damaged atrial septum, further engagement with the septal wall can be undesirable and result in further damage to the patient. Furthermore, in some patients, the septal wall may be occupied with one or more implant devices or other treatments, wherein it is not tenable to traverse the septal wall in view of such treatment(s). As alternatives to transseptal access, transaortic access may be implemented, wherein a delivery catheter is passed through the descending aorta 32, aortic arch 12, ascending aorta, and aortic valve 7, and into the left atrium 2 through the mitral valve 6. Alternatively, transapical access may be implemented to access the target anatomy.

The process 900 may involve placing a guidewire 54 in the left atrium 2 via a pathway through the coronary sinus. For example, such guidewire placement may involve one or more of the operations associated with blocks 902, 903, and 904, which, as with any other of the blocks of the process 900, may be considered optional operations. The operations associated with blocks 902, 903, and 904 relate to steps in making a puncture hole through a wall of the coronary sinus for placement of a shunt implant device in accordance with aspects of the present disclosure between the coronary sinus 16 and left atrium 2, wherein the associated images of FIG. 10-2 show views of the relevant anatomy as seen looking down on a section of the heart with the posterior aspect down.

Any of several access pathways visible and/or apparent in the image 1001 may be implemented for maneuvering guidewires and delivery systems/catheters in and around the heart to implement any of the operations associated with the various blocks of the process 900. For instance, access may be from above via either the subclavian or jugular veins into the superior vena cava 19, right atrium 5 and from there into the coronary sinus 16. Alternatively, the access path may start in the femoral vein and through the inferior vena cava 29 into the heart. Other access routes may also be used, and each typically utilizes a percutaneous incision through which a guidewire and/or catheter are inserted into the vasculature, normally through a sealed introducer, and from there the physician controls the distal ends of the devices from outside the body.

At block 902, the process 900 involves placing a guidewire 54 in the coronary sinus 16. For example, as mentioned, the guidewire 54 may be introduced through the subclavian or jugular vein, through the superior vena cava 19 and into the coronary sinus 16. Since the coronary sinus 16 is largely contiguous around the left atrium 2, there are a variety of possible acceptable placements for a shunt implant in accordance with the present disclosure. The site selected for placement of the shunt implant (and therefore for puncture through the tissue wall 21), may be made in an area where the tissue of the particular patient is less thick or less dense, as determined beforehand by non-invasive diagnostic means, such as a CT scan or radiographic technique, such as fluoroscopy or intravascular coronary echo. Image 1002 shows the guidewire 54 being advanced from the right atrium 5 into the coronary sinus 16 through its ostium or opening 14.

At block 903, the process 900 involves puncturing the tissue wall 21 to provide access into the left atrium 2. Image 1003 shows a puncture catheter 91 that has been advanced over the guidewire 54. The puncture catheter 91 can be introduced into the body through a proximal end of an introducer sheath (not shown). The introducer sheath can be configured to provide access to the particular vascular pathway (e.g., jugular or subclavian vein) utilized, and may have one or more hemostatic valves associated therewith/therein. While holding the relevant introducer sheath at a fixed location, the surgeon can manipulate the puncture catheter 91 to the implant site.

At least a distal end of the puncture catheter 91 may have a slight curvature built/formed therein, so as to conform to the curved coronary sinus. An expandable anchoring member 92 can be exposed/projected along a radially-outer side of the catheter 91, such as in a region adjacent an extreme distal segment 94 that may be thinner than or tapered narrower from the proximal extent of the catheter. One or more radiopaque markers may be disposed on the catheter 91 to help the surgeon determine the precise advancement distance for desired placement of the anchoring member 92 within the coronary sinus.

Image 1003 shows radially-outward deployment of the expandable anchoring member 92, which may comprise a bulbous balloon (as shown), a braided mesh, or other feature. One advantage of a mesh is that it can avoid excessive blockage of blood flow through the coronary sinus during the procedure, though the procedure typically does not take very long and a balloon can be preferrable in some instances. Expansion of the anchoring member 92 can serve to press the radially inner curve of the catheter 91 against the luminal wall 21 of the coronary sinus. Consequently, a needle port opposite the balloon anchor 92 can be pressed to abut the luminal wall 21. The positioning of the anchor 92 may advantageously orient the puncturing of a puncture needle 93 through the needle port approximately above the “P2” portion of the posterior leaflet of the mitral valve 6.

The puncture sheath/needle 93 may include a sharp distal tip and may be advanced along the catheter 91 such that it exits the needle port at an angle from the longitudinal direction of the catheter and punctures through the wall 21 into the left atrium 2. The puncture sheath needle 93 can be curved in a manner as to orient the needle toward the left atrium 2 when deployed from the catheter 91.

At block 904, the process 900 involves inserting a guidewire 59 into the left atrium 2 through the puncture path created by the puncture sheath 93. For example, the needle tip may be retracted/removed from the puncture sheath 93, thereby providing an open a lumen within the puncture sheath 93 through which a second guidewire 59 can be advanced into the left atrium 2. In some instances, the guidewire 59 may pass through a lumen provided in the puncture needle within the puncture sheath 93. The puncture sheath 93 may then be removed from the left atrium and into the puncture catheter 91, leaving the guidewire 59 extending through the coronary sinus and into the left atrium.

At block 905, the process 900 involves dilating the puncture/opening formed in the tissue wall 21. Image 1005 shows a dilator 95 that may be advanced along the guidewire 59 and at least partially through the tissue wall 21 into the left atrium 2. The dilator 95 may comprise, for example, an elongated inflatable balloon. The dilator 95 and an inflation tube may ride over the guide wire 59, and may be held in place during retraction of the catheter 91, which may be retracted some amount to avoid interference with the dilator. The dilator 95 may be radially expanded, such as through inflation, so as to widen the puncture through the tissue wall 21. The dilator 95 can then be retracted into the puncture catheter 91, after which the puncture catheter 91 may be removed along the guide wire 59. Although separate puncture and implant delivery catheters/systems are illustrated and described, it should be understood that shunt implant devices in accordance with the present disclosure may be implanted using a procedure in which puncturing a hole between the coronary sinus and the left atrium as well as delivering the shunt are accomplished with a single access device/system.

At block 906, the process 900 involves providing a delivery system 51 with a shunt implant device 70 disposed therein in a delivery configuration, such as a sensor-equipped shunt implant device as disclosed in detail herein. Image 1006A of FIG. 10-4 shows a partial cross-sectional view of a delivery system 51 for a shunt implant device 70 in accordance with one or more embodiments of the present disclosure. The image 1006A shows the shunt implant device 70 disposed within an outer sheath 58 of the delivery system 51. Although a particular embodiment of a delivery system is shown in FIG. 10-4, it should be understood that shunt implant devices in accordance with aspects of the present disclosure may be delivered and/or implanted using any suitable or desirable delivery system and/or delivery system components.

The illustrated delivery system 51 includes an inner catheter/shaft 55, which may be disposed at least partially within the outer sheath 58 during one or more periods of the process 900. In some embodiments, the shunt implant device 70 may be disposed at least partially around the inner catheter/shaft 55 and at least partially within the outer sheath 58 during one or more periods of the process 900. For example, the inner catheter 55 may be disposed within the barrel portion 78 of the shunt implant 70, as shown.

The shunt implant 70 can be disposed about a shunt-holder portion/component 80 of the inner catheter/system 55. Image 1006B shows a detailed view of the shunt-holder portion 80, which may be integrated with the inner catheter/shaft 55, or may be a separate component of the system 51 that is attached or otherwise coupled to the inner catheter 55 in some manner. In some embodiments, the shunt holder 80 includes a sensor-holder/accommodation means 83 including one or more cut-outs, indentations, recesses, channels, gaps, openings, apertures, holes, slits, or other features configured to accommodate the presence of the sensor device 76 and/or other feature(s) or aspect(s) of the implant device 70. For example, the sensor device 76 may be disposed at least partially within an inner diameter of the shunt structure 70 in the delivery configuration shown in image 1006A. In such configurations, the sensor assembly component(s) may create an interference with respect to the ability of the shunt 70 to be disposed relatively tightly around the shunt holder portion 80, thereby potentially increasing the profile of the delivery system 51 and/or affecting the ability of the shunt implant device 70 to be delivered using the delivery system 51. Therefore, as shown in FIG. 10-3, the shunt holder 80 may include one or more sensor-accommodation features, such as a sensor recess/channel 83, shown in images 1006A and 1006B. The sensor-accommodation recess/channel 83 may comprise a longitudinal and circumferential space/cut-out of the shunt holder 80. The accommodation feature 83 may advantageously be dimensioned to correspond to the size and/or profile of the sensor device 76, as shown.

The sensor-accommodation recess/channel 83 may be configured to have the sensor device 76 disposed at least partially therein, which may be a cylindrical sensor device as described above. By including/forming the sensor recess/channel 83 in the shunt holder 80, which provides an open space within the radial area/boundary of the shunt holder 80 and/or inner catheter/shaft 55, the delivery system 51 can allow for transport of the shunt implant device 70 along with the sensor device 76, which may be coupled to the shunt implant device 70 in some manner as described in detail above prior to transport, without the bulkiness of the sensor device 76 undesirably increasing the radial profile of the shunt implant device 70 in the compressed/crimped delivery configuration shown in image 1006A substantially beyond the radial profile of the inner catheter 55 and/or shunt holder 80. For example, with the barrel 78 of the shunt 70 crimped/compressed, as shown, the sensor device 76 may be positioned to nest and/or otherwise be disposed at least partially within the radial and longitudinal space of the sensor recess/channel 83. Therefore, the shunt implant device 70 may be able to be transported within the delivery system 51, wherein the outer sheath 58, which may present the outer radial boundary/profile of the delivery system 51, is able to maintain a relative/narrow profile, which may be necessary or desirable for delivery through various blood vessels of the patient's vasculature.

During delivery, the shunt implant 70 may be positioned on the inner catheter 55 and/or shunt holder portion 80 in a manner such that a base 77 of the sensor device 76 and/or a sensor holder tab/structure coupled to the base 77 of the sensor device rests on and/or contacts an axial stopper surface/feature 82 of the shunt holder portion 80. The shunt holder 80 may further include various other features to assist in delivery of the shunt implant device 70 to the target implantation site. For example, in some embodiments, the sensor holder 80 includes a guidewire channel 84, which may comprise a lumen/channel that extends longitudinally/axially through a portion of the shunt holder 80 and/or inner catheter 55. The guidewire channel 84 may have a distal opening through which the guidewire 59 can extend, wherein the shunt holder 80 and/or inner catheter 55 may be configured to slide over the guidewire 59 as necessary to execute the process 900 process. The shunt holder 80 can further include one or more additional channels and/or openings for navigating sutures through the inner catheter 55 for securing and/or manipulating the shunt 70. For example, a proximal opening 81 may lead to a channel 85 within the shunt holder 80 and/or inner catheter 55, wherein one or more sutures or suture portions 56 may be passed through the opening 81 and around/through one or more features of the shunt 70, such as through the apertures of the proximal/short anchor arms 53 of the shunt 70. The suture connection to the proximal anchor arms 53 can facilitate deployment of the arms 53, as described below.

The sensor recess/channel 83 may allow for the barrel 78 of the shunt 70 to be deflected radially inwardly with respect to a circumferential portion of the barrel 78 associated with the sensor device 76. That is, generally when compressing/crimping the barrel 78, the struts of the shunt 70 may deflect axially/longitudinally to reduce the diameter of the barrel for transportation in a reduced-diameter configuration, as shown in image 1006A. In the area of the sensor holder 79, the barrel 78 may be deflected radially inward to create a circumferential bend/corrugation that allows the sensor 76 to further encroach into the radial area of the barrel 78, thereby reducing the radial profile of the combined shunt and sensor assembly. For example, image 1006A shows the barrel 78 with an inward radial bend 75 on either side of the sensor 76. The longitudinal surface of the channel/recess 83 may be concave in some embodiments, wherein such concavity can accommodate the cylindrical shape of the sensor housing. Alternatively, the surface 87 of the sensor recess/channel 83 can be substantially flat.

In some embodiments, the shunt holder 80 includes a distal peg feature 86, which may be utilized to couple and/or mate the shunt holder 80 with a nosecone feature 52. Such feature 86 may serve to hold the shunt holder 80 in a secure position during transport. In some embodiments, a distal portion 88 of the shunt holder 80 may taper radially outward to fill the space between the shunt holder 80 and the outer sheath 58, thereby providing increased stability for such components.

As referenced above, the delivery system 51 may include a tapered nosecone feature 52, which may be associated with a distal end of the sheath 58, catheter 55, and/or delivery system 51. In some implementations, the nosecone feature 52 may be utilized to further dilate the opening in the tissue wall 21 and/or guide the delivery system 51 through the opening without damaging the tissue. The nosecone feature 52 may further facilitate advancement of the distal end of the delivery system 51 through the tortuous anatomy of the patient and/or within an outer delivery sheath or other conduit/path. The nosecone 52 may be a separate component from the shunt holder 80 and/or inner catheter 55, or may be integrated with either component. In some embodiments and/or stages, the nosecone 52 can be disposed adjacent to and/or integrated with a distal end of the outer sheath 58. In some embodiments, the nosecone 52 may comprise and/or be formed of multiple flap-type forms that can be urged/spread apart when the shunt implant device 70 and/or any portions thereof, the shunt holder 80, the interior catheter 55, or other device(s) are advanced therethrough.

The shunt implant device 70 can be positioned within the delivery system 51 with a first end thereof that is associated with the distal anchor arm(s) 54 disposed distally with respect to the barrel 78 of the shunt structure 70. A second end of the implant 70 associated with the proximal anchor arm(s) 53 is positioned at least partially proximally with respect to the barrel 78 of the shunt structure 70 and/or the sensor device 76.

The outer sheath 58 may be used to transport the shunt implant device 70 to the target implantation site. That is, the shunt implant device 70 may be advanced to the target implantation site at least partially within a lumen of the outer sheath 58, such that the shunt implant device 70 is held and/or secured at least partially within a distal portion of the outer sheath 58.

In the compressed delivery configuration shown in image 1006A, the shunt 70 may have a generally tubular form. In such a configuration, the distal 54 and proximal 53 anchor features/arms can form extensions of the barrel walls 78 in a tubular form. For example, the tubular shape may correspond to a further-crimped configuration of the shape that the shunt 70 may have immediately after being laser cut from a tubular workpiece. During manufacturing, the un-crimped tubular form of the shunt 70 may be deformed using mandrels and the like to bend the anchor features/arms radially outward into the expanded configuration shown, for example, in FIGS. 3A-3F. The shunt 70 in its deformed shape may then be heat treated such that the memory metal (e.g., nitinol) material reaches a transition temperature and the expanded shape becomes the relaxed/programmed shape of the shunt 70. The memory metal shunt can then be bent into a tubular shape and crimped to decrease the diameter of the tube for loading within the delivery catheter.

At block 907, the process 900 involves advancing the delivery system 51 including the shunt implant device 70 into the coronary sinus and to the target implantation site. Image 1007 shows the introduction of the shunt deployment or delivery catheter/system 51, which may have a soft, tapered distal tip/nosecone 52, as described above. The system 51 may be advanced along the guide wire 59 that remains bridging the tissue wall 21 between the coronary sinus 16 and the left atrium 21.

At block 908, the process 900 involves accessing, with the delivery system 51, the left atrium 2 through the opening formed in the tissue wall 21. Image 1008 shows the delivery catheter 51 advanced through the puncture in the tissue wall 21 into the left atrium 2, which passage can be facilitated by dilation of the puncture as described above and the soft, tapered distal tip/nosecone 52. The delivery catheter 51 is shown with a transparent outer sheath 58 to illustrate the expandable shunt 70 therein, just proximal to the distal tip 52. The expandable shunt 70 is shown in the crimped, generally tubular configuration described above, which facilitates passage through the lumen of the outer sheath/catheter 58.

At block 909, the process 900 involves deploying the relatively long distal anchor arms 54 of the shunt 70 on the left atrial side of the tissue wall 21. Image 1009 shows the long distal anchor arms 54 expanded within the left atrium 2 into contact with the tissue wall 21. This expansion can be initiated by retraction of the outer sheath 58 of the delivery catheter/system 51 relative to the inner catheter 55 and shunt holder portion 80. The shunt 70 is disposed at this time in the annular space between the inner shunt holder 80 and outer sheath 58. The shunt holder 80 passes through a central flow passage of the shunt 70. The shunt 70 is collapsed/crimped into a compressed tubular configuration with the anchor arms 54 thereof straightened, wherein the arms 54 are configured to spring open when the restraining outer sheath 58 retracts. Radiopaque markers may be associated with the arms 54 to facilitate positioning immediately within the left atrium 2. The sensor device 76 is deployed at least partially within the opening 1095 in the tissue wall, between the barrel 78 and the inner diameter of the tissue wall opening 1095.

At block 910, the process 900 involves deploying the proximal anchor arms 53 of the shunt 70 on the coronary sinus side of the tissue wall 21. The proximal arms 53 may be deployed using certain actuator components (e.g., actuating rod(s)) associated with the delivery system 51, or may automatically deploy through their shape-memory characteristics. For example, eyelet/aperture features of the anchor arms can be provided for gripping by a rod or other actuator feature of the system 51, wherein engagement of the aperture feature can allow for actuation/manipulation of the anchor arms 53 into the desired deployed position. Image 1010 shows the deployment of the proximal arms, wherein either or both of primary 1091 and secondary 1092 tissue contact pads of the proximal arms 53 may be deployed on the coronary sinus side of the wall 21 and/or within the opening 1095 in the tissue wall 21.

Deployment of the proximal/short anchor arms 53 can be implemented at least in part by the physician retracting the outer sheath 58 further proximally relative to the shunt holder 80 and/or further withdrawing the shunt holder 80 back through the opening 1095 until the two distal relatively short proximal anchor arms 53 and/or tissue contact pads associated therewith come into contact with the tissue wall 21. This can be felt by tactile feedback, or by once again confirming the position of the anchor arm(s) by radiopaque visualization.

In some implementations, prior to full deployment of the proximal anchor arms 53 as shown in image 1010, the proximal anchor arms 53 may be retained by one or more sutures or suture portions 56, which may be looped through the primary 1091 and/or secondary 1092 tissue contact pads of the proximal arms 53 to prevent the proximal arms 53 from expanding prematurely. Once the proximal arms 53 are believed to be positioned within the opening 1095, but prior to their release from the delivery catheter 51, a contrast injection may be made in the vicinity to see whether the shunt is properly positioned. Once released from the delivery system 51, the proximal anchor arms/pads 53 are permitted to resiliently contact the tissue wall 21 (or at least the luminal surface of the coronary sinus or the inner diameter/surface of the opening 1095).

At block 911, the process 900 involves withdrawing the delivery system 51. thereby leaving the shunt implant device deployed in the tissue wall 21 and shunting blood from the left atrium 2 into the coronary sinus 16. Included in such withdrawal is the guidewire 59. which is also removed from the patient's anatomy. The primary retention mechanism for the shunt 70 comes from the geometrical constraint of the design-the length and contact area/dimensions of the distal anchor arms 54, which prevent the shunt 70 from being pulled through the opening 1095. Furthermore, the shunt 70 is retained at least in part by a radial force exerted outward on the tissue wall opening 1095 from the barrel 78 of the shunt 70. The opposed clamping forces of the proximal anchor arms 53 also help hold the shunt 70 in place. With the implant 70 fully deployed, elevated left atrial pressure can thus be ported through the implanted shunt 70 into the coronary sinus 16 as indicated by the arrow in image 1011.

Although described in the context of implanting the shunt 70 in the wall between the coronary sinus and left atrium, it should be understood that the process 900 may be implemented, at least in part, to implant the shunt 70 in other anatomy and/or tissue walls, such as the atrial septum or ventricular septum. The shunt 70 may also be positioned between other cardiac chambers, such as between the pulmonary artery and right atrium.

### Additional Description of Examples

Provided below is a list of examples, each of which may include aspects of any of the other examples disclosed herein. Furthermore, aspects of any example described above may be implemented in any of the numbered examples provided below.

Example 1: A shunt device comprising a cylindrical barrel portion formed of a plurality of struts arranged in a chevron pattern, a plurality of proximal anchor features emanating from a first axial end of the barrel portion, and a plurality of distal anchor arms emanating from a second axial end of the barrel portion, the plurality of distal anchor arms having a length that is greater than a length of the proximal anchor features.

Example 2: The shunt device of any example herein, in particular example 1, wherein each of the plurality of proximal anchor features comprises a primary tissue contact pad and a secondary tissue contact pad circumferentially and axially offset from the primary tissue contact pad.

Example 3: The shunt device of any example herein, in particular example 2, wherein the primary tissue contact pad and the secondary tissue contact pad of each of the plurality of proximal anchor features are coupled by a strut that is bowed away from the barrel portion.

Example 4: The shunt device of any example herein, in particular any of examples 1-3, wherein the barrel portion includes a plurality of wishbone struts coupled between secondary tissue contact pads of the plurality of proximal anchor features.

Example 5: The shunt device of any example herein, in particular any of examples 1-4, wherein the shunt device is configured to be deployed in a tissue wall such that the shunt device holds the tissue wall between the plurality of proximal anchor features and the plurality of distal anchor arms with the barrel portion disposed at least partially in an opening in the tissue wall.

Example 6: The shunt device of any example herein, in particular any of example 5, wherein the barrel portion has an axis that is angled relative to a plane of the tissue wall when the shunt device is deployed in the tissue wall.

Example 7: The shunt device of any of any example herein, in particular any of examples 1-6, wherein the plurality of distal anchor arms each include an elongated strut terminating in a tissue-contact pad.

Example 8: The shunt device of any example herein, in particular of example 7, wherein at least a portion of the elongated strut and tissue-contact pad of each of one or more of the plurality of distal anchor arms is covered with a covering configured to promote tissue-ingrowth.

Example 9: The shunt device of any of any example herein, in particular any of examples 1-8, further comprising a sensor holder emanating from the first axial end of the barrel portion.

Example 10: The shunt device of any example herein, in particular of example 9, wherein the sensor holder comprises a ring configured to deflect outwardly from the barrel portion and have a base of a cylindrical sensor device attached thereto such that the sensor device is positioned on an outside of the barrel portion.

Example 11: The shunt device of any of any example herein, in particular any of examples 1-10, wherein the plurality of distal anchor arms are circumferentially offset from the plurality of proximal anchor features.

Example 12: The shunt device of any of any example herein, in particular any of examples 1-11, wherein the plurality of proximal anchor features are dimensioned to be deployed in a coronary sinus of a patient when the shunt device is deployed in a wall separating the coronary sinus from a left atrium with the plurality of distal anchor arms deployed in the left atrium.

Example 13: The shunt device of any of any example herein, in particular any of examples 1-12, wherein the shunt device is sterilized.

Example 14: A shunt device comprising a cylindrical barrel formed of a plurality of struts arranged in a zigzag pattern, a plurality of first tissue contact pads associated with a first axial end of the barrel and configured to deflect radially outward from an axis of the barrel, and a plurality of second tissue contact pads associated with a second axial end of the barrel and configured to deflect radially outward and toward an axial center of the barrel, the plurality of second tissue contact pads being angularly offset from the plurality of first tissue contact pads about the axis of the barrel.

Example 15: The shunt device of any example herein, in particular of example 14, wherein the plurality of first tissue contact pads and one of the plurality of second tissue contact pads are positioned on a first diametrical side of the shunt device, and wherein two of the plurality of second tissue contact pads are positioned on a second diametrical side of the shunt device.

Example 16: The shunt device of any example herein, in particular of example 15, wherein one of the plurality of second tissue contact pads is angularly positioned between the plurality of first tissue contact pads:

Example 17: The shunt device of any example herein, in particular of example 15 or example 16, further comprising a sensor holder associated with the first axial end of the barrel on the second diametrical side and angularly positioned between the two of the plurality of second tissue contact pads.

Example 18: The shunt device of any example herein, in particular of example 17, wherein the sensor holder is coupled to a plurality of third tissue contact pads via a plurality of wishbone struts that are open axially away from an axial center of the barrel.

Example 19: The shunt device of any example herein, in particular of example 18, wherein each of the plurality of third tissue contact pads is coupled to a respective one of the plurality of first tissue contact pads via a connecting strut.

Example 20: The shunt device of any example herein, in particular of example 18 or example 19, wherein one or more of the plurality of third tissue contact pads comprises an echogenic marker.

Example 21: The shunt device of any of any example herein, in particular any of examples 14-20, wherein one or more of the plurality of second tissue contact pads is covered by a cloth sock.

Example 22: The shunt device of any of any example herein, in particular any of examples 14-21, wherein the shunt device is sterilized.

Example 23: A shunt device comprising a barrel configured to hold open an opening in a tissue wall and formed of a plurality of columns of wishbone struts including a first set of wishbone struts open in a first axial direction and a second set of wishbone struts open in a second axial direction opposite the first axial direction, a plurality of proximal anchoring means associated with a first axial end of the barrel and configured to contact a first side of the tissue wall when the barrel is disposed in the opening in the tissue wall, and a plurality of distal anchoring means associated with a second axial end of the barrel and configured to contact a second side of the tissue wall opposite the first side when the barrel is disposed in the opening in the tissue wall.

Example 24: The shunt device of any example herein, in particular of example 23, wherein at least some of the first set of wishbone struts and the second set of wishbone struts are coupled at apices thereof to respective longitudinal struts of the barrel.

Example 25: The shunt device of any example herein, in particular of example 23 or example 24, wherein at least some of the first set of wishbone struts and the second set of wishbone struts are not coupled at apices thereof to a longitudinal strut of the barrel.

Example 26: The shunt device of any of any example herein, in particular any of examples 23-25, wherein the plurality of proximal anchoring means each comprise a primary tissue contact pad coupled to a secondary tissue contact pad via a curved connecting strut.

Example 27: The shunt device of any example herein, in particular of example 26, wherein the secondary tissue contact pad of each of the plurality of proximal anchoring means is longitudinally and angularly offset from the primary tissue contact pad of a respective one of the plurality of proximal anchoring means with respect to an axis of the barrel.

Example 28: The shunt device of any of any example herein, in particular any of examples 23-27, wherein the plurality of distal anchoring means each comprise an elongate arm with a distal tissue contact pad.

Example 29: The shunt device of any of any example herein, in particular any of examples 23-27, wherein at least some of the first set of wishbone struts or second set of wishbone struts form closed chevron cells.

Example 30: The shunt device of any of any example herein, in particular any of examples 23-29, wherein the shunt device is sterilized.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering Therefore, as used herein, an ordinal term (e g., “first,” “second.” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A shunt device comprising:
a cylindrical barrel portion formed of a plurality of struts arranged in a chevron pattern;
a plurality of proximal anchor features emanating from a first axial end of the barrel portion; and
a plurality of distal anchor arms emanating from a second axial end of the barrel portion, the plurality of distal anchor arms having a length that is greater than a length of the proximal anchor features.

2. The shunt device of claim 1, wherein each of the plurality of proximal anchor features comprises a primary tissue contact pad and a secondary tissue contact pad circumferentially and axially offset from the primary tissue contact pad.

3. The shunt device of claim 2, wherein the primary tissue contact pad and the secondary tissue contact pad of each of the plurality of proximal anchor features are coupled by a strut that is bowed away from the barrel portion.

4. The shunt device of claim 1, wherein the barrel portion includes a plurality of wishbone struts coupled between secondary tissue contact pads of the plurality of proximal anchor features.

5. The shunt device of claim 1, wherein the shunt device is configured to be deployed in a tissue wall such that the shunt device holds the tissue wall between the plurality of proximal anchor features and the plurality of distal anchor arms with the barrel portion disposed at least partially in an opening in the tissue wall.

6. The shunt device of claim 5, wherein the barrel portion has an axis that is angled relative to a plane of the tissue wall when the shunt device is deployed in the tissue wall.

7. The shunt device of claim 1, wherein the plurality of distal anchor arms each include an elongated strut terminating in a tissue-contact pad.

8. The shunt device of claim 7, wherein at least a portion of the elongated strut and tissue-contact pad of each of one or more of the plurality of distal anchor arms is covered with a covering configured to promote tissue-ingrowth.

9. The shunt device of claim 1, further comprising a sensor holder emanating from the first axial end of the barrel portion.

10. The shunt device of claim 9, wherein the sensor holder comprises a ring configured to deflect outwardly from the barrel portion and have a base of a cylindrical sensor device attached thereto such that the sensor device is positioned on an outside of the barrel portion.

11. A shunt device comprising:
a cylindrical barrel formed of a plurality of struts arranged in a zigzag pattern;
a plurality of first tissue contact pads associated with a first axial end of the barrel and configured to deflect radially outward from an axis of the barrel; and
a plurality of second tissue contact pads associated with a second axial end of the barrel and configured to deflect radially outward and toward an axial center of the barrel, the plurality of second tissue contact pads being angularly offset from the plurality of first tissue contact pads about the axis of the barrel.

12. The shunt device of claim 11, wherein the plurality of first tissue contact pads and one of the plurality of second tissue contact pads are positioned on a first diametrical side of the shunt device, and wherein two of the plurality of second tissue contact pads are positioned on a second diametrical side of the shunt device.

13. The shunt device of claim 11, wherein one of the plurality of second tissue contact pads is angularly positioned between the plurality of first tissue contact pads.

14. A shunt device comprising:
a barrel configured to hold open an opening in a tissue wall and formed of a plurality of columns of wishbone struts including a first set of wishbone struts open in a first axial direction and a second set of wishbone struts open in a second axial direction opposite the first axial direction;
a plurality of proximal anchoring means associated with a first axial end of the barrel and configured to contact a first side of the tissue wall when the barrel is disposed in the opening in the tissue wall; and
a plurality of distal anchoring means associated with a second axial end of the barrel and configured to contact a second side of the tissue wall opposite the first side when the barrel is disposed in the opening in the tissue wall.

15. The shunt device of claim 14, wherein at least some of the first set of wishbone struts and the second set of wishbone struts are coupled at apices thereof to respective longitudinal struts of the barrel.

16. The shunt device of claim 14, wherein at least some of the first set of wishbone struts and the second set of wishbone struts are not coupled at apices thereof to a longitudinal strut of the barrel.

17. The shunt device of claim 14, wherein the plurality of proximal anchoring means each comprise a primary tissue contact pad coupled to a secondary tissue contact pad via a curved connecting strut.
